(19) United States
(12) Patent Application Publication
(10) Pub. No.: US 2008/0077430 A1
(43) Pub. Date: Mar. 27, 2008

(54) Systems and methods for improving medication adherence

(76) Inventors: Michael S. Singer, Cambridge, MA (US); Murat V. Kalayoglu, Boston, MA (US)

Correspondence Address: FISH & RICHARDSON PC, P.O. BOX 1022, MINNEAPOLIS, MN, 55440-1022, US

(21) Appl. No.: 11/527,169

(22) Filed: Sep. 25, 2006

(51) Int. Cl. G06Q 10/00; A61B 5/00

(52) U.S. Cl. 705/2; 600/300

(57) ABSTRACT

The invention relates to simple yet effective systems and methods for inducing patients to take their medications, without the need for outside prompting, and without the need for electronic medication dispensers. Instead, the new systems and methods use simple medication dispensers with unique markings to motivate the patients to take their medications based on voluntary actions to obtain a reward, e.g., an intermittent reward. In addition, the new systems and methods allow the collection of user data, all volunteered by the users.

### TECHNICAL FIELD

This invention relates to systems and methods for improving medication adherence, also known as medication compliance.

### BACKGROUND

Each year, medication non-adherence leads to 125,000 deaths in the U.S. Half of all medication-related hospital admissions (10% of all hospitalizations) are attributed to poor compliance, at an annual cost of $100 billion to the healthcare system. In addition, with typical compliance rates of only about 50%, pharmaceutical companies lose some $100 billion annually in potential sales.

Non-compliance can occur with any disease but is particularly common in asymptomatic conditions such as hypertension and diabetes. This is because patients rarely perceive any benefit from the medication. For example, half of those patients started on a “statin” (HMG-COA reductase inhibitor) stop within 6 months. Patients are broadly distributed on the spectrum from absolutely non-compliant to perfectly compliant, and a given patient's compliance may fluctuate over time. There is often a disparity between true compliance levels and what patients report to their physicians and researchers. Perhaps counter-intuitively, non-compliance does not correlate with age, gender, race, education, or income.

Experts have classified non-compliance into three basic types. The first, most common type, is erratic non-compliance, which involves patients who understand instructions, but miss doses due to forgetfulness or other priorities. The second type, unwitting non-compliance, involves patients who do not understand how or when to use their medication. The third type, rational non-compliance, involves patients who believe, erroneously or not, that their own chosen patterns of medication use are better than those recommended by their doctor.

Many methods to improve compliance have been described. See, e.g., Osterberg et al., “Adherence to Medication,” N. Engl. J. Med., 353:487-497 (2005). These methods include: (1) reminders and alarms; (2) patient education; (3) complicated electronic medication dispensers; and (4) an improved patient-doctor relationship. These methods are typically costly and labor-intensive. Most of these methods are “passive” in the sense that they act upon the patient rather than having the patient take an “active,” voluntary role in their own improved compliance. Unfortunately, studies have shown these existing methods of improving compliance to be only minimally effective.

### SUMMARY

The invention relates to simple yet effective systems and methods for inducing patients to take their medications, without the need for outside prompting, and without the need for electronic medication dispensers. Instead, the new systems and methods use simple medication dispensers with unique markings to motivate the patients to take their medications based on voluntary actions to obtain a reward, e.g., an intermittent reward. In addition, the new systems and methods allow the collection of user data, all volunteered by the users.

The invention also features new methods of Dynamic Intermittent Reward (DIR), which are used in combination with the new systems and methods to provide individual users with random or nonrandom intermittent rewards, but allow system operators to control overall costs and cost-effectiveness of the reward program. DIR offers rapid, sensitive, and tailored methods to boost a desired behavior, such as taking a prescribed medication.

In addition, the invention features new methods of Tabular Intermittent Reward (TIR), which are used in combination with the new systems and methods to provide individual users with intermittent rewards on a predetermined yet interesting schedule. TIR makes it possible to administer rewards in a fashion theoretically predictable to the user and without any element of chance. Under some circumstances the TIR approach has particular legal and social advantages.

In general, in one aspect, the invention features medication dispensers that are associated with a dispenser identification code and include, on an outer or inner surface a set of markings from which a user can derive a reward code.

In some embodiments, the medication dispensers include a container designed to contain one or more medication doses and have a first set of markings; a movable component including a second set of markings and configured to move in relationship to the container to enable a user to align one or more markings of the first set of markings with one or more markings of the second set of markings and to enable the user to derive a reward code from one or more of the markings; and a dispenser identification code associated with the dispenser.

These dispensers can further include a user identification code associated with the dispenser. The user identification codes can be an alphanumeric code stored on an electromagnetic device or on an electronic apparatus readable medium. Both the dispenser and user identification codes can be or include a series of letters, numbers, or symbols, or any combination of none, one, or more of letters, numbers, and symbols. The use identification code can be a user's telephone number or part of the telephone number. The user identification code can be comprised of one or more of the markings in the first or second sets of markings, or in both sets of markings. The dispensers can be used to dispense medication doses in the form of, e.g., individual tablets, solid forms, liquid aliquots, transdermal patches, gases, semi-solids, or powders.

In certain embodiments, the container can be a bottle including an opening and the movable component can include a cap configured to seal the bottle, and the first and second sets of markings can be arranged so that they can be aligned by rotating the cap in relation to the bottle when the cap is placed over the opening of the bottle. In these dispensers the first and second sets of markings can be arranged so that when the cap is in a closed position, no marking on the bottle is aligned with a marking on the cap, and so that a marking in the first set can be aligned with a marking in the second set only when the cap is in an open position.

In other embodiments, the dispensers can have a container in the form of a blister pack that includes a plurality of blisters each containing one or more medication doses. In these embodiments, the movable component can be, for example, slideably attached to the blister pack such that the component can slide along a linear path in relation to the blister pack. In other embodiments, the movable component can be rotatably attached to the blister pack such that it rotates around an axis of the blister pack.

The invention also features dispensers in which the container is disc-shaped and has the first set of markings arranged around an axis. In these devices, the movable component can include a shell configured to rotate about the container and cover a portion of the container. The shell has the second set of markings arranged around the axis, and the first and second sets of markings can be arranged so that they can be aligned by rotating the shell in relation to the container. In some examples, one or more of the markings on the container are covered by the shell when the dispenser is in a closed position. The medication doses can be inhalation doses of a medication in powder, liquid, or gaseous form, or the medication doses can be drops of an ophthalmic or nasal solution or suspension.

In any of these dispensers, the markings in the first set can be numbers and the markings in the second set can be letters, or vice versa. The first and second sets of markings can each include a random series of letters, numbers, or iconic symbols, or any combination of none, one, or more of letters, numbers, and iconic symbols. In some embodiments, e.g., for use with children or adults who cannot read letters or symbols, the first and second sets of markings, or both, can include a series of differently colored markings.

In another aspect, the invention features medication dispensers that include a container; a movable component including one or more markings and configured to move in relation to the container to dispense a medication dose and to display the one or more markings after the dose has been dispensed to enable a user to derive a reward code from one or more of the markings; and a dispenser identification code associated with the dispenser. For example, these dispensers can be used to dispense medication doses in the form of measured liquid aliquots. In these dispensers one or more of the markings on the movable component can be covered by a portion of the container before the dose is administered. In certain embodiments, this type of dispenser can include a syringe body and the movable component can include a plunger. In some examples, the container includes a window through which one or more of the markings are visible after the dose is administered. The dispenser can be configured to dispense a single medication dose per actuation.

In other embodiments, the new medication dispensers can be designed to be associated with a dispenser (and/or user) identification code and to contain one or more medication doses in solid form, wherein one or more of the medication doses in the container includes one or more markings from which a user can derive a reward code. Thus, the medication doses, e.g., pills, capsules, or tablets, themselves can be provided with markings that the user can observe after taking the pills out of the container. The markings may themselves be the reward code, and only some of the pills in the container may be marked. Alternatively, all of the pills may be marked, and only some of the markings are, or contain, reward codes.

In another aspect, the invention features methods for improving medication adherence by a user by obtaining the dispenser identification code via a communications network to identify a medication dispenser that comprises a reward code (e.g., within a series of markings); providing instructions to the user via the communications network to observe and/or manipulate the medication dispenser and determine a reward code, e.g., based on one or more markings derived from one or more sets of markings on the dispenser; obtaining the reward code from the user via the communications network; and providing the user with a reward report, wherein the reward report indicates whether a reward will be awarded, and if so, a reward value. The reward can be awarded on an intermittent basis. The methods can also include providing an automated contact center that a user can contact via the communications network and that provides automated instructions and the reward report to the user. In certain embodiments, the methods also include providing to the user a medicine dispenser associated with a dispenser identification code and including one or more sets of markings, e.g., one of the dispensers described herein.

In these methods, the instructions can include directions to align a first mark in the first set of markings (e.g., a specific letter in a series of letters) on the movable component with a second mark in the second set of markings (e.g., a specific number in a series of numbers) on the container and to determine a reward code including a third mark aligned with a fourth mark (e.g., a letter aligned with a number or vice versa). In addition, in some embodiments when the user contacts the automated contact center with a telephone, the user's telephone number can include or be the user identification code, and the automated contact center can automatically determine the identity of the user based on the telephone number.

In another aspect, the invention features methods for improving medication adherence by identifying a medication user; obtaining the user's medication compliance history for a specific medication; optionally calculating a medication compliance rate over a specific period of time based on the medication compliance history; determining whether a reward should be provided for a given compliance event as a function of the user's compliance history or rate; and, if a reward is to be provided, determining a reward value. The methods can also include providing the user with a reward report indicating whether a reward will be awarded and if so, the reward value. These methods can further include providing to the user a medicine dispenser associated with a user identification code and obtaining the user identification code via a communications network to identify the user; and obtaining and storing a medication compliance history for the user and the specific medication. These methods do not need to use the medication dispensers described herein.

In some embodiments of these methods, the user has to contact the center within a designated time period to be eligible to obtain a reward report. In certain embodiments, the reward value, a frequency of a reward being awarded, or both can be a function of the user's medication compliance history or compliance rate. For example, the reward value, the frequency of a reward being awarded, or both can be positively or inversely correlated with an improvement in the user's medication compliance history or compliance rate. An improvement can be calculated, for example, based on a difference between a present medication compliance event and a medication compliance event that occurred at an earlier time period, e.g., from 1 hour to 1 month, before a present time period. The reward value, the frequency of the reward being awarded, or both can be periodically recalculated. For example, they can be recalculated every 8 hours, 12 hours, 24 hours, 48 hours, or 7 days.

In any of the methods described herein, the function can include a random or pseudorandom expression, and the reward value, the frequency of the reward being awarded, or both can be selected from among a plurality of predetermined values, and each predetermined value can be associated with a particular level of medication compliance; e.g., wherein each predetermined value is associated with a particular medication compliance rate. Alternatively, or in addition, the reward value, the frequency of the reward being awarded, or both can be defined by a constant minus the compliance rate (or other measure of the compliance history) or by a constant divided by the compliance rate (or other measure of the compliance history), of a specific user over a specified period of time.

In addition, in any of the methods described herein the reward value, the frequency of the reward being awarded, or both can be defined such that, when correlated with a compliance rate (or other measure of the compliance history) of the user, the Spearman's rank correlation coefficient is less than 0 (e.g., −0.3, −0.5, −0.7), or is equal to −1.0.

In another aspect, the invention features systems for improving medical adherence that include any of the medication dispensers described herein; and a contact center that includes a communications port, a processor, and an electronic apparatus readable medium configured to cause the processor to: obtain the dispenser identification code via a communications network to identify the dispenser; provide instructions to the user via the communications network to observe the medication dispenser and determine a reward code including one or more markings derived from the one or more sets of markings; obtain the reward code from the user via the communications network; and provide the user with a reward report, wherein the reward report indicates whether a reward will be awarded.

In these systems, the electronic apparatus readable medium can be further configured to cause the processor to determine whether the user is communicating with the contact center at an appropriate time and/or to cause the processor to determine whether a reward is to be awarded based on an intermittent reward algorithm or any of the other algorithms described herein. As in the new methods, the reward value, frequency of a reward being awarded, or both can be a function of the user's medication compliance history or compliance rate and the functions can be defined as described and claimed herein for the various methods.

In certain embodiments a third party, such as a health care provider, can determine the medical compliance of a patient. The electronic apparatus readable medium can be further configured to cause the processor to report to the user that a medication refill will soon be needed or the systems can automatically transmit a refill request to a medication dispensary, e.g., upon patient approval.

As used herein, a user identification code or dispenser identification code is “associated with” a medication dispenser when the code is connected to the dispenser either physically, e.g., directly or indirectly, or electronically, e.g., in a computer database or a magnetic card (or other machine-readable storage medium) that is provided to the user along with the dispenser. The code is connected indirectly to the dispenser when the two are provided to a user together or for use together. For example, a medication dispenser may be provided to a user along with a tag or card (e.g., plastic or paper) that includes the code. In another example, the medication dispenser may be provided to the user along with a card or other device that includes an electronic, machine-readable storage medium that contains the user identification code.

Alternatively, the user may already have such a card that includes the user identification code, and the card is updated with a new dispenser identification code to indicate that a new medication dispenser has been provided to the user. Alternatively, the user may have one user identification code that is associated with all of the user's medication dispensers for a given period of time, e.g., a month, 3 months, 6 months, or even a year or more. In this scenario, the code can be provided to the user either printed on a card or stored electronically, e.g., at the beginning of the given time period. The user code can be provided by the distributor of the medication dispensers, e.g., a pharmacy, and can be the pharmacy's code for that patient. A single user may have one user identification code for all of his or her different medications, a different code for each medication, a different code for each dispenser, or a different code for each refill of a medication. However, each dispenser must have a unique dispenser identification code or at least a unique combination of a user identification code and dispenser code.

As used herein, a “set of markings” means any series of symbols, characters on a computer keyboard, e.g., letters or numbers, or other unique indicia recognizable by either a person or a computer, or both. The term “symbol” refers, inter alia, to any iconic character or colored shape. The term “indicia” means, inter alia, any letter, number, or character on a computer keyboard. Some markings may be considered a symbol or an indicium, and thus the terms symbol and indicia can in some instances be used interchangeably.

As used herein, a “reward code” is a single marking or series of markings, e.g., a single letter, number, or symbol, or a series of letters, numbers, and/or symbols, or combinations thereof, that a user can derive from one or more markings or sets of markings on a medication dispenser. The reward code is analyzed or evaluated, e.g., by a central control system (e.g., an automated contact center), to determine whether the user will obtain a reward for a given medication compliance event according to the methods described herein. Reward codes can also be derived from one or more markings or sets of markings printed, etched, or imprinted on the medication doses, e.g., pills, capsules, or tablets, themselves.

As used herein, the term “derived from” as used to describe how a user determines a reward code from the one or more sets of markings on a medication dispenser means that the user observes and/or manipulates the medication dispenser as instructed by the contact center and then observes one or more specific symbols or indicia within the one or more sets of markings on the medication dispenser, that form the reward code. For example, in the case of a dispenser that includes symbols on one portion of the dispenser and indicia on a second portion, the user may move, e.g., rotate or slide, one portion with respect to the other to align a certain symbol with a certain indicium. Then the user is instructed to observe a first specific symbol or indicium and indicate a second specific indicium or symbol aligned opposite the first. The second symbol or indicium can be the reward code.

A “medication compliance (or adherence) history” is a record of two or more medication compliance events, or a lack of expected compliance events (e.g., based on prescription information), for a given user. A compliance event is a contact by a user to a control center, e.g., an automated contact center, to provide to the center a reward code derived from a medication dispenser during an appropriate time period (e.g., one, two, three, or even six or more hours before or after a prescribed time to take a given medication).

As used herein, a “medication compliance (or adherence) rate” can be calculated as the number of compliance events for a given medication dispenser divided by the total number of expected compliance events based on a prescription for the specific medication dispenser.

The invention provides numerous advantages including the direct, e.g., automatic (or personal) interaction with patients on a daily basis, the collection of volunteered information during the daily interactions, simple reward modifications at any time, and the use of a simple, low-cost, intuitive medication dispenser, such as a pill bottle, that requires no batteries, activation, or electronics of any kind. In addition, the new systems and methods work with any medications, either branded or generic, that require a repeated administration over several days, weeks, months, or even years. The new systems and methods are also easy for any pharmacy or other distributor of medication dispensers to implement, and can operate in cooperation with or in parallel with the distributor's existing patient codes and automated refill systems.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the description and drawings, and from the claims.

### DESCRIPTION OF DRAWINGS

FIG. 1 is a schematic diagram of an overview of the new system of improving medication compliance.

FIG. 2 is a schematic block diagram illustrating the components of a system employing one embodiment of the present invention.

FIG. 3 is a flow chart that shows the general steps of the new systems and methods.

FIGS. 4A to 4D are different views of a new medication dispenser in the form of a pill bottle marked for use in the new systems and methods.

FIG. 5 is a flow chart that shows the general steps of the new systems and methods when used with a pill bottle of FIGS. 4A to 4D.

FIGS. 6A to 6C are different views of a new medication dispenser in the form of a canister inhaler.

FIGS. 7A to 7C are different views of a new medication dispenser in the form of a disk-shaped inhaler.

FIGS. 8A to 8F are different views of new medication dispensers in the form of medication blister packs.

FIGS. 9A and 9B are different views of a new medication dispenser in the form of a syringe.

FIG. 10 is a flow chart illustrating an embodiment of the new Dynamic Intermittent Reward system.

FIG. 11 is a schematic diagram of an exemplary database table for user (patient) information.

FIG. 12 is a schematic representation of an exemplary database of adherence records for a patient.

FIG. 13 is an exemplary Static Table for use in Tabular Intermittent Reward

FIG. 14 is an exemplary table of adherence statistics for a patient.

FIG. 15 is a schematic block diagram illustrating exemplary components of an automated contact center of the system of FIG. 2.

FIG. 16 is a schematic diagram of an automated contact center and its connection to multiple users and multiple types of access devices to communicate with the users.

FIG. 17 is a graph that shows the mean adherence rates for each day of the Control (diamonds) or Intervention (circles) phase of a clinical study.

FIG. 18 is a bar graph that shows mean adherence rates for each participant in the Control phase (dark bars) and the Intervention phase (white bars) of the clinical study of FIG. 17.

FIG. 19 is a bar graph showing a 33% relative improvement in compliance resulting from use of the new systems and methods.

Like reference symbols in the various drawings indicate like elements.

### DETAILED DESCRIPTION

The invention relates to systems and methods of improving medication compliance using simple, but unique medication dispensers that include dispenser and/or user identification codes along with other markings, e.g., symbols and/or indicia. Patients can contact a control center, e.g., an automated contact center, each time they take their medication, and use the medication dispensers to obtain a code they provide to the contact center to determine whether they will obtain a reward. In certain embodiments, the invention includes the use of Dynamic Intermittent Rewards (DIR) to better control or tailor the rewards to specific users and to lower the overall cost of managing a given system. In some embodiments, the invention includes the use of Tabular Intermittent Rewards (TIR) to render a given system predictable to the user and avoid an element of chance. In other embodiments, the systems can use the new medication dispensers with an intermittent reward system without the use of DIR or TIR, or even with a continuous reward system.

### General Methods and Systems for Improving Medication Compliance

The concept underlying the new systems and methods is quite simple. Medications come dispensed in unique medication dispensers (described in more detail below) that each include a dispenser identification code, e.g., a unique dispenser identification code, and can also include a user identification code that is unique to each patient in the system. The dispensers also include one or more sets of markings, e.g., symbols and/or indicia, that are printed on one or more parts of the dispenser itself or on a label that is typically attached to such medication dispensers.

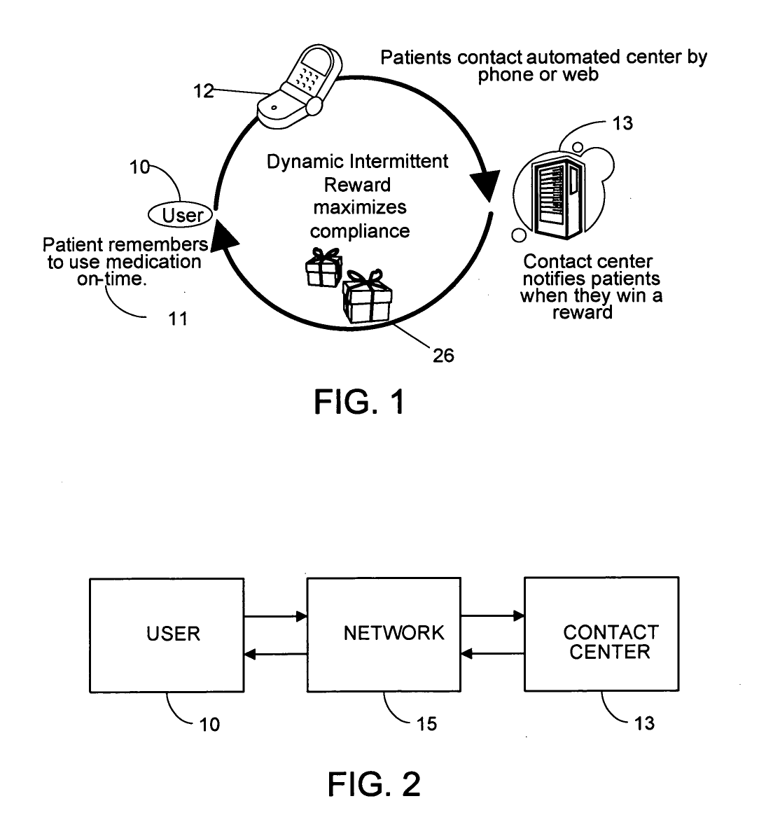

New medications may come with a printed invitation, e.g., a tag or card, that includes a statement such as “Want to obtain free rewards just for taking your pills on time? Just call 1-555-555-5555.” With each scheduled dose the patient is motivated to contact a contact center, e.g., an automated contact center, for example, using a toll-free telephone number or a website, for the opportunity to obtain a reward. The contact center can also be run by one or more human operators. For example, Mr. John Sample (user 10) is instructed by a physician to take a pill twice daily. As shown in the schematic diagrams of FIGS. 1 and 2, he remembers to take the pill within the correct time frame (step 11) and contacts (step 12) an automated contact center 13 using a communication network 15, e.g., a telephone system, a computer-based system such as the Internet, an intranet, or a local area network (“LAN”) or wide area network (“WAN”), e.g., within a hospital, e.g., by email or “live chat.” The communication network can also be wireless, permitting contact by cellular telephone, walkie-talkie, and other radio or infrared frequency devices. The contact center 13 obtains the dispenser identification code and optionally a user identification code from the user via the communication network to identify the dispenser and optionally the user as well.

The contact center 13 then provides instructions to the user 10 to observe and/or manipulate the medication dispenser and determine a reward code comprising one or more letters, numbers, or symbols, or any combination of none, one, or more of letters, numbers, and symbols derived from the one or more sets of markings. Next, the user provides the reward code to the system via the communication network; and the system provides the user with a reward report. The reward report recites an intermittent reward, i.e., a reward is awarded on an intermittent schedule and/or on a constant or intermittent schedule with a variable amount. Thus, the reward may be intermittent with a fixed or varying value, or constant (e.g., a reward for each call) with a varying value, e.g., the value could be a chance to obtain a larger reward, e.g., a lottery ticket, or a small value such as one dollar, or one or more “points” or coupons, which can be combined with other points or coupons to obtain a larger reward.

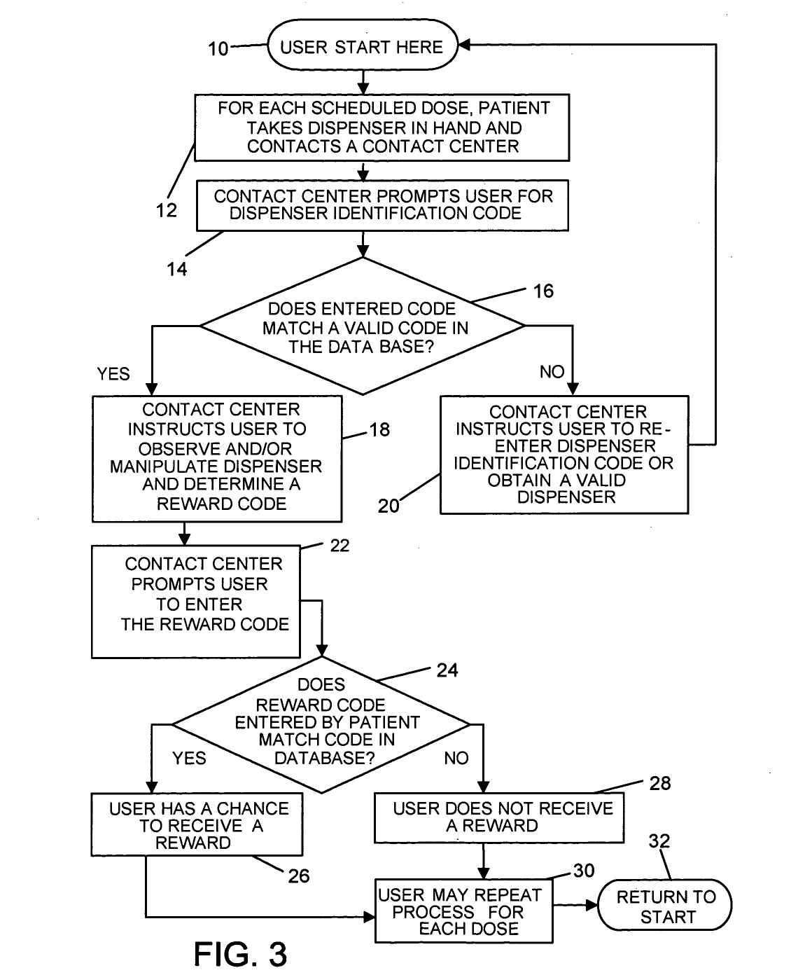

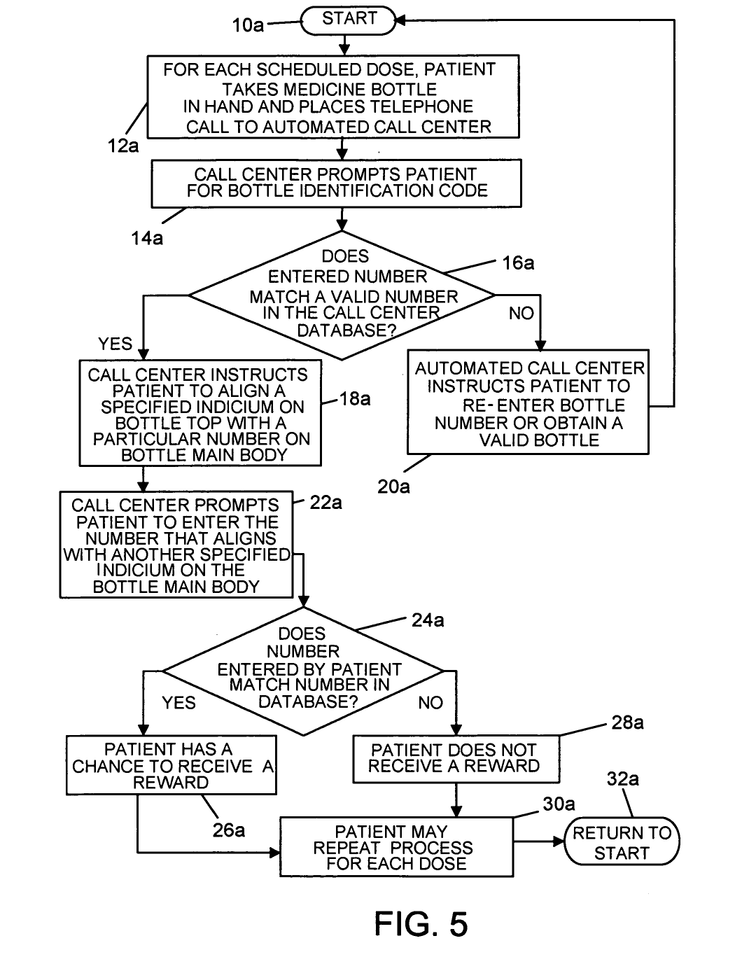

In one embodiment, as shown in the flow chart of FIG. 3, the user 10 remembers to take the pill within the correct time frame and contacts a contact center (step 12), e.g., by telephone or computer, e.g., by email or “live chat.” The center requests a dispenser identification code, e.g., number (step 14) and if the dispenser code is valid (step 16) instructs the user to inspect or manipulate the medication dispenser and determine a reward code (step 18; see also, FIG 5D, step 18a). If the dispenser code is not valid, the center asks the user to re-enter the code or to obtain a valid dispenser (step 20).

If the dispenser code is valid, the contact center then asks the user to indicate the reward code, e.g., by entering a number into a telephone keypad or computer keyboard, or just by saying the correct sequence of numbers, letters, and/or symbols (step 22). The contact center then determines whether the reward code entered matches a valid reward code in a database (step 24). If the reward code matches one of those in the database, the contact center tells the user whether he has obtained a reward (step 26) according to an intermittent reward, Dynamic Intermittent Reward, or Tabular Intermittent Reward algorithm, as described in further detail below. In other words, the user does not always obtain a reward, and the reward value can vary from each compliance event to the next. If the patient does not obtain a reward (step 28), he has another opportunity to contact the center during the time period for the next dose (step 30), and the system returns to a starting mode (step 32).

Rewards can vary from a small value, e.g., a $3 gift card to a particular store, e.g., a coffee shop, to a large value, e.g., free airline tickets or a vacation package. Since most rewards involve gift cards, it may be necessary to deliver a card to the user only once. Afterwards the contact center can add value to the cards electronically. The contact center may be automated and programmed with the proper dosage windows for each medication dispenser. Thus, patients cannot contact the center too often or off-schedule. If they try to contact the center at an incorrect time, they will be told to take their medication and contact the center at the next appointed time .

The new methods also provide for automated refills to help ensure that medications are refilled on time. When it is time to refill the medication, the automated contact center notifies the user and offers to send a refill request to the pharmacy. In some embodiments, the user must approve the refill request.

### Medication Dispensers

The new methods and systems can be used in conjunction with a wide variety of medication dispensers. These dispensers must all include a dispenser identification code that is either unique to that dispenser (to distinguish that dispenser from all other dispensers), or provides a unique code combination when combined with a particular user identification code or other identifying information (e.g., the user's telephone number). For example, all dispensers for a particular drug may have the same dispenser code (thereby simplifying manufacture of the label or other container marking), but each user has a different user code, so the combination of dispenser code and user code is unique.

These dispenser identification codes can be printed onto a label that is applied to a medication dispenser either by a pharmacist who dispenses the medication to the patient, by a distributor, or by the manufacturer when the medication is packaged. Alternatively, the code can be printed on a tag or card that is associated with, e.g., distributed along with, the dispenser. Many high-volume drugs are repackaged at pharmacies, but others are pre-packaged by the manufacturer in units suitable for dispensing to a patient (for example, birth control pills, Zithromax® Z-paks, and many HIV medications). The new systems and methods are designed to work equally well with medication dispensers filled by pharmacists or pre-filled by the manufacturer. In certain embodiments, the codes can be printed or marked directly onto a component of the dispensers. For dispensers used by pharmacists, the dispensers can be provided to the pharmacists already marked with the codes. The pharmacists then merely need to add a standard medication label to the pre-marked dispenser in the same way they presently label dispensers.

In certain embodiments, the information for contacting the automated center, e.g., a call center phone number, listed on the dispenser (or associated with, e.g., provided with, the dispenser) can function as part of the dispenser identification code. For example: ID 64 58 72, please call 888 555 6677 versus ID 64 58 72, please call 866 555 3458. These two different telephone call-in numbers can be used to differentiate the two different dispensers (e.g., one telephone number for all dispensers for a specific drug, and a second telephone number for all dispensers that contain a second specific drug), yet allow the system to keep the identification code associated with the dispenser to a manageable, e.g., four-, five-, or six-digit, number (of course, larger numbers are possible). The identification code could also be based upon the pharmacy's prescription number or symbols and/or indicia from the dispenser as discussed in further detail below.

In addition, the dispensers can include a user identification code that is unique to a given patient. The user identification code is added to the dispenser, e.g., on a label or sticker that can be affixed to the dispenser, by the pharmacist when he or she packages the medication for the patient. Alternatively, the user code can be a unique, random alphanumeric code applied to the dispenser in advance, e.g., by a third party or by the dispenser manufacturer, and then associated with a specific patient by the pharmacist when dispensing the medication. The pharmacist can associate the random code with a specific patient or user by linking the code with the patient's contact information in a computer database or the patient can associate himself or herself with a particular dispenser code during contact with the automated contact center, which links the user identification code with the user contact information and the dispenser code in computer databases.

Alternatively, the pharmacy may already have a unique patient identification number, and the new systems can use that number as the user identification code, and can also use the contact information that pharmacies typically have associated with their patient identification numbers.

In yet another embodiment, the automated contact center can generate a unique user identification code the first time the user contacts the center and can link the user code with the first dispenser code and any subsequent dispenser codes used by the same user. The methods of linking information in different databases can be carried out using any standard database manipulation software.

In certain embodiments, when a patient first picks up a new dispenser from a pharmacy, the automated contact center database may not have a link between a new user identification code and the dispenser identification code. Nor need the system burden the pharmacist with establishing that link. Instead, the system will automatically establish the link the first time (or a subsequent time) the patient calls and enters the new dispenser identification code. For example, the system, detecting that the dispenser has not been used before, can announce to the patient, e.g., “If this is a new bottle, please press 2 now.” The system then prompts the patient for his or her user identification code, which can be listed on a membership card or otherwise obtained from the pharmacist or other source. Alternatively, the system can detect the caller's identification based on the caller's telephone number (“caller ID”) and use this telephone number as the user identification code. In another alternative, the system can generate a new user identification code and link that code to the dispenser and the home address and other contact information provided by the patient. In this way, the system links every dispenser to a particular person and optionally their contact information, and can track that person's behavior (e.g., medication adherence and choices of rewards) over time.

The new dispensers must also include one or more sets of markings, e.g., symbols and/or indicia, on one or more components of the dispenser. Some dispensers have no moving parts, and then may have just one set of markings. Some dispensers have a container, e.g., a bottle or canister, and a moving component, such as a lid, cap, or shell, that can be moved with respect to the container. These dispensers can have one set of markings, e.g., symbols or indicia, on the container, and a second set of markings on the movable component. The markings can be on the outside of, e.g., in plain view on, a container or movable component or on the inside or otherwise obscured when the dispenser is closed.

The one or more sets of markings do not necessarily have any meaning that a patient can derive by observing them, but once instructed by the automated contact center, the patient can derive a reward code from the individual symbols, characters, numbers, letters, colors, and/or other indicia in the markings. The user can then provide the reward code to the automated contact center to find out whether he or she will obtain a reward and the value of the reward.

The symbols can be iconic symbols, e.g., [symbol] , [symbol] , [symbol] , [symbol] , [symbol] , [symbol] , ▴, ♡, [symbol] , [symbol] , [symbol] , [symbol] , ¤, [symbol] , [symbol] , and [symbol] . The symbols can also be any shapes of different sizes and marks of different colors. The indicia can be any letters, e.g., capitol or lower case, numbers, or other typewriter keyboard symbols, such as @, #, $, %, &, {, >, !, +, or *. Thus, the indicia can be any alphanumeric string of characters. As used herein, the symbols and indicia as listed on the medication dispensers can be used in combination or interchangeably. For example, a set of symbols can include indicia, and vice versa.

The medication dispensers fall into at least three categories. First, the dispensers can have a container and a movable component, in which each part has a separate set of markings. This type of dispenser includes pill bottles, disk-shaped inhalers, standard inhalers, eyedrop bottles, and blister packs. Second, the dispensers can have one container with or without a separate movable component, but in which only the container or the movable component includes a set of markings.

Third, the new medication dispensers can be designed to be associated with a dispenser (and/or user) identification code and to contain one or more medication doses in solid form, wherein one or more of the medication doses in the container includes one or more markings from which a user can derive a reward code. Thus, the medication doses, e.g., pills, capsules, or tablets, can themselves be provided with markings that the user can observe after taking the pills out of the container. The markings may themselves be the reward code, and only some of the pills in the container may be marked. Alternatively, all of the pills may be marked, and only some of the markings are, or contain, reward codes (e.g., each pill may have a series of several numbers, letters, or symbols, and only one of these markings, e.g., the middle of three letters, is the reward code). The pills may be marked or etched by a laser to form the markings, or may be imprinted with indentations to form the marks, or the marks may be printed onto the surface of the pills, e.g., using a pharmaceutically acceptable printing ink. All of these methods of marking pills, tablets, or capsules are known in the art.

Pill Bottle

In one embodiment, the medication dispenser is a standard pill bottle that has a container with an opening and a cap or lid that can either screw onto or snap onto the container. Either way, the cap can be rotated with respect to the container.

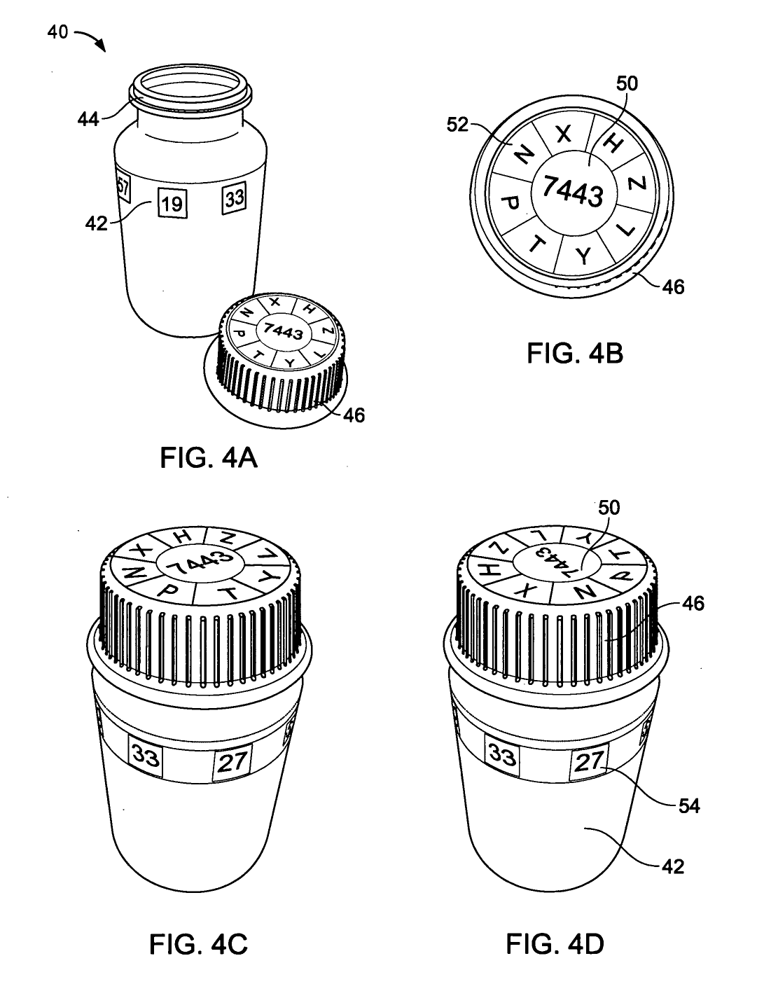

FIGS. 4A to 4D depict various views of such a pill bottle 40 with a body 42 and an opening 44, and its related markings. As shown, cap 46 can rotate such that the letters 52 depicted on the cap align with various numbers 54 on the main label of the container 42. The cap in this example also includes a dispenser identification code 50, but this code can be on the container 42. In this example, the patient can rotate the cap to any of five positions. This allows the patient to read particular letter-number combinations, such as “P-33” (FIG. 4C). The process of turning the cap to a given position not only builds anticipation for a reward, but also stimulates the patient to open the pill bottle.

When instructed by the system, the user rotates the cap to align the cap markings with the container markings in a certain way, e.g., aligning the “X” on the cap with the “33” on the container. Then the user is asked to observe another one of the cap markings, e.g., the “N” and determine which number now is aligned with the N. In FIG. 4D, the “27” is aligned with the N. The user then indicates to the system that the reward code is 27, and waits to learn if he or she has obtained a reward. The cap and container can be designed such that none of the markings on the cap and the container are aligned when the bottle is closed, to ensure that the patient has to open or at least rotate the cap to be able to make the requested alignment.

FIG. 5 shows a flow chart similar to FIG. 3 and indicates the steps the system takes when a user contacts the automated contact center. As shown in the chart, the user 10a remembers to take the pill within the correct time frame, takes the pill bottle in hand, and contacts an automated contact center (step 12a), e.g., by telephone or computer, e.g., by email or “live chat.” The center requests a bottle identification code (step 14a) and if this code is valid (step 16a) instructs him to turn the cap on his pill bottle to a particular position (step 18a; example: “Line up the letter X with the number 33”). This orientation of the cap reveals particular letter-number combinations from which the user can derive a reward code. If the bottle identification code is not valid, the center asks the user to re-enter the code or to obtain a valid pill bottle (step 20a).

If the bottle identification code is valid, the call center then asks the user to indicate the reward code, e.g., by entering a number into a telephone keypad or computer keyboard, or just by saying the number (step 22a). The contact center then determines whether the reward code entered matches a valid reward code in a database (step 24a). If the reward code matches one of those in the database, the contact center tells the user whether he has obtained a reward (step 26a) according to an intermittent schedule or Dynamic Intermittent Reward algorithm, as described in further detail below. In other words, the user does not always obtain a reward, and the reward value can vary from each compliance event to the next. If the patient does not obtain a reward (step 28a), he has another opportunity to contact the center during the time period for the next dose (step 30a), and the system returns to a starting mode (step 32a).

Canister Inhaler

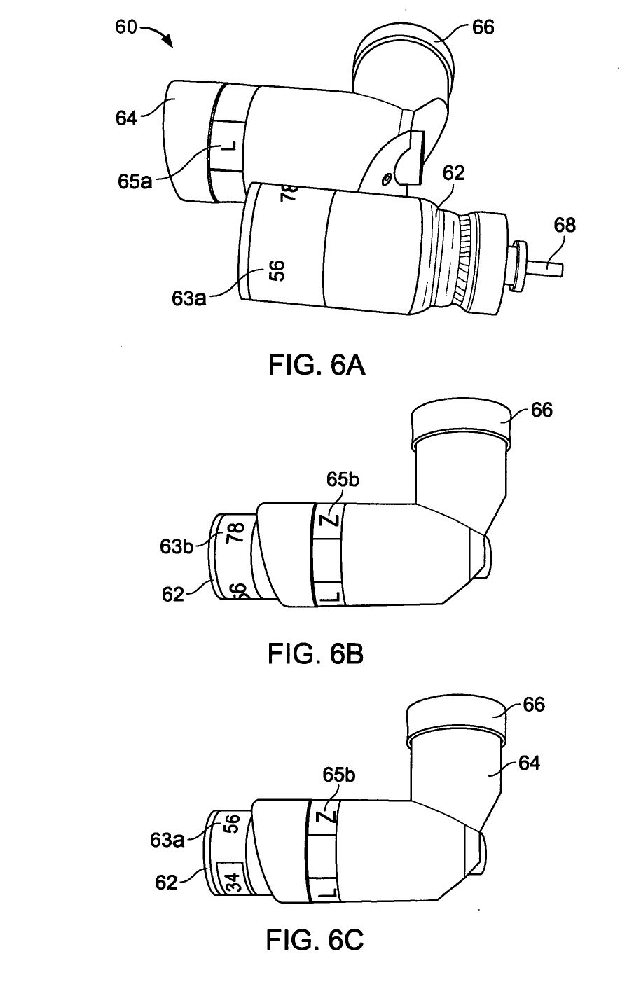

As shown in FIGS. 6A to 6C, the medication dispenser can be in the form of a standard canister inhaler 60, e.g., a metered-dose inhaler such as those used for asthma, chronic obstructive pulmonary disease, cystic fibrosis, and other pulmonary diseases. A metered-dose inhaler 60 can also be used to administer insulin to a patient by the inhaled route. A metered-dose inhaler typically comprises an inhaler body 64, a mouthpiece 66, and a reservoir (e.g., a canister) 62 of medication to be inhaled. The medication may be suspended with propellants and stored in a canister under pressure. Alternatively, medication may be stored as a dry powder or a liquid solution, with the medication propelled from the inhaler by mechanical force or suction. The construction of inhalers and reservoirs and the preparation of medication and propellants are familiar to any skilled artisan.

If the reservoir is a canister 62, it is fitted with a medication outlet 68, typically a tubular male component sized to fit securely into a complementary female receptacle inside the inhaler body 64. The fit between the medication outlet and receptacle is sufficiently secure that the medication canister remains attached to the inhaler body during normal use. At the same time, the medication outlet 68 has rotational freedom such that the medication canister 62 can be rotated around the axis of the medication outlet (and thereby with respect to the inhaler body 64) and still remain securely attached to the inhaler body. This characteristic is put to new use in the present invention.

When the inhaler in actuated, a metered dose of medication is released from the medication canister and channeled from the medication outlet to the receptacle of the inhaler body. This receptacle is fashioned to direct or redirect the spray of medication out of the mouthpiece 66. The patient inhales the medication from the mouthpiece and into the lungs. Similar inhalers may be used to deliver medications to the nose; such inhalers are particularly useful for delivering steroids, decongestants, or peptides such as calcitonin.

As shown in FIG. 6A, the canister 62 is marked with a series of numbers (63a), whereas the movable inhaler body 64 is marked with a series of letters (65a). As shown in FIG. 6B, the number “78” (reference 63b), is initially aligned with the letter “Z” (65b). In FIG. 6C, the body 64 has been rotated with respect to the canister 62 so that the letter “Z” (65b) is now aligned with the number “56” (reference 63a). The patient can then determine that the letter “L” is aligned with the number “34.” Thus, if the patient is instructed to find the code that aligns with the letter “L,” the reward code can be indicated as “34.”

Disc-Shaped Inhaler

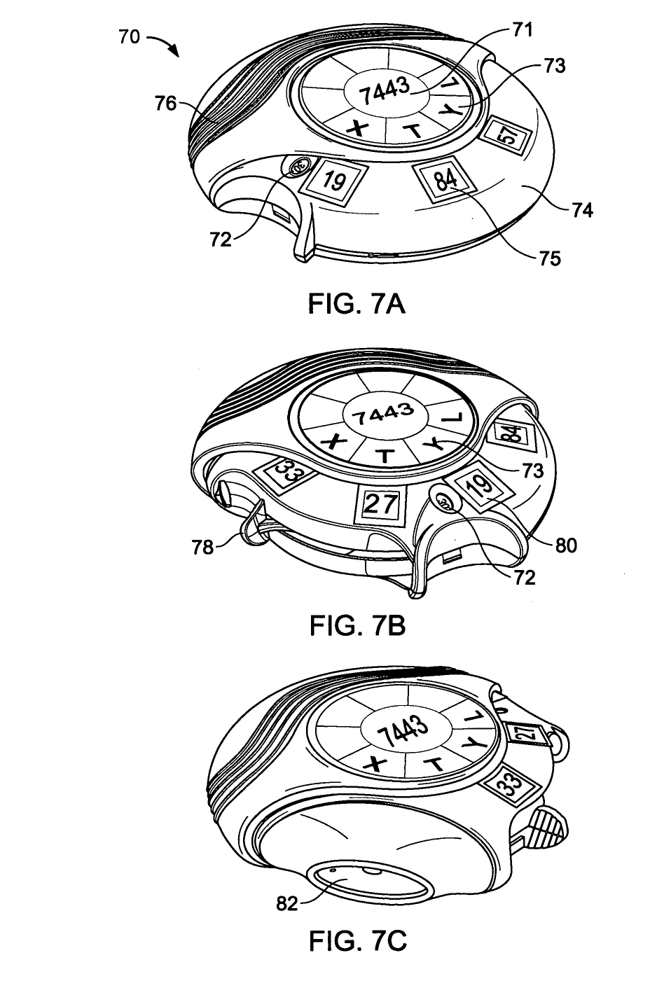

As shown in FIGS. 7A to 7C, the medication dispenser can be in the form of a disk-shaped inhaler 70. Similar to the canister inhaler, the disk-shape inhaler holds medication in a reservoir, for example in dry powder form, and delivers the medication in into the lungs of a patient. Such inhalers are may be used for asthma, chronic obstructive pulmonary disease, cystic fibrosis, other pulmonary diseases, or diabetes.

The disk-shaped inhaler 70 comprises an inhaler body 74, a mouthpiece 82, a reservoir (internal, not shown), an optional numerical dose counter 72, and a movable shell 76. An advancement lever 78 positions a new metered dose for use. The numerical dose counter 72 indicates either the number of doses already administered or the number of doses remaining. The construction of such inhalers and reservoirs and the preparation of medication are familiar to any skilled artisan.

When the inhaler in actuated, a metered dose of medication is released from the medication reservoir and channeled from the mouthpiece 82 and into the patient's lungs, and the numerical counter, if present, is incremented.

As shown in FIG. 7A and FIG. 7C, the inhaler body 74 is marked with a series of numbers (75 and 80), whereas the movable shell 64 is marked with a series of letters (73). The movable shell is shown marked optionally with a dispenser identification number 71. As shown in FIG. 7A, the shell is in a “closed” position with respect to the inhaler body, whereby the mouthpiece is covered. In this state the number “84” (reference 75), is initially aligned with the letter “T”. In FIG. 7B, the shell is in a “partially open” position, and the letter “Y” (73) is now aligned with the number “19” (reference 80). In this state the patient can determine that the letter “T” is aligned with the number “27.” Thus, if the patient is instructed to find the code that aligns with the letter “T,” the reward code can be indicated as “27.” In FIG. 7C, the shell is in the “fully open” position, whereby the mouthpiece 82 is fully exposed.

Blister Pack

The medication dispenser can be in the form of a blister pack with an outer cover, sleeve, or envelope that can move with respect to the blister pack, either by rotating about an axis or by sliding in relationship to the blister pack. The format and production of blister packs are obvious to anyone skilled in the art. They are typically fashioned as a piece of plastic with one or more concavities; each of which may hold one or more pills or other discrete medication forms; each of which is covered with a suitable layer of foil, paper, or plastic; and each of which may be ruptured by a patient to collect the medication inside.

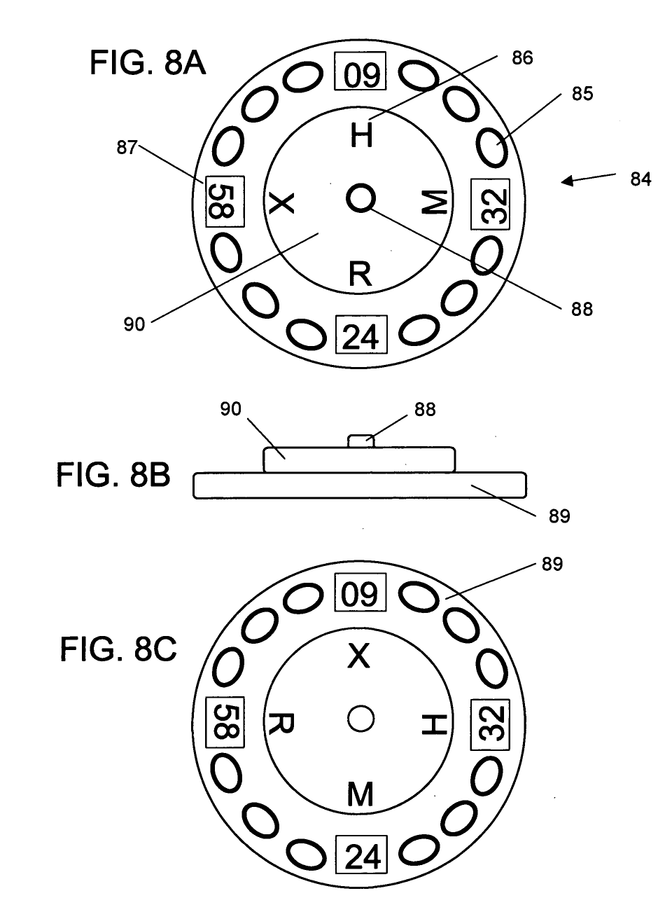

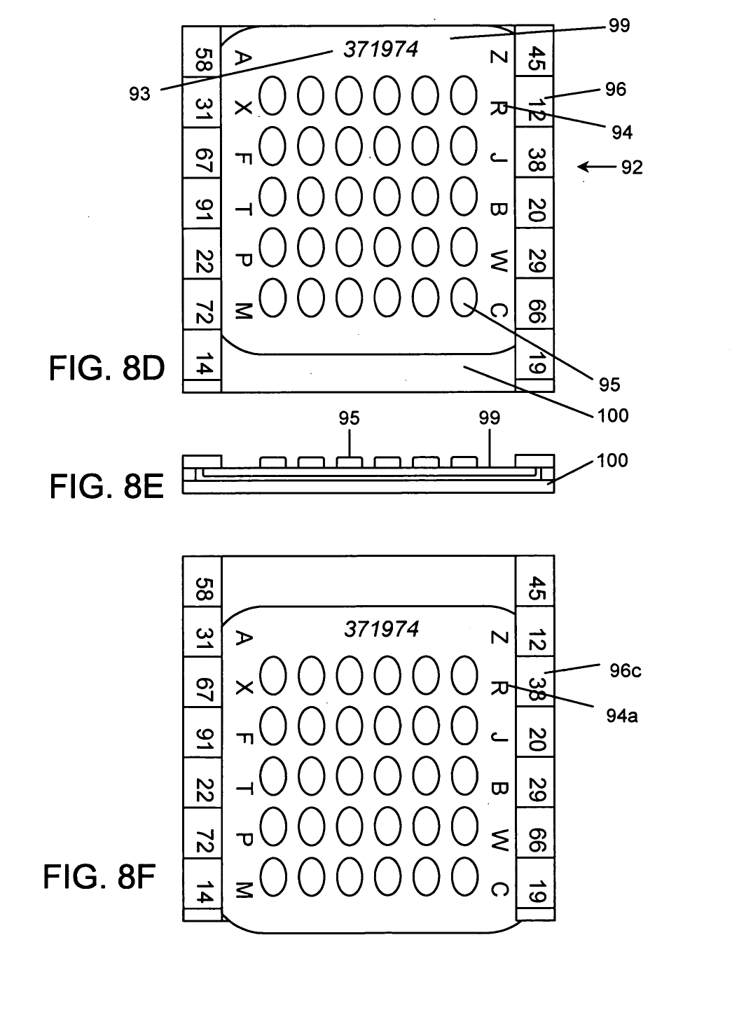

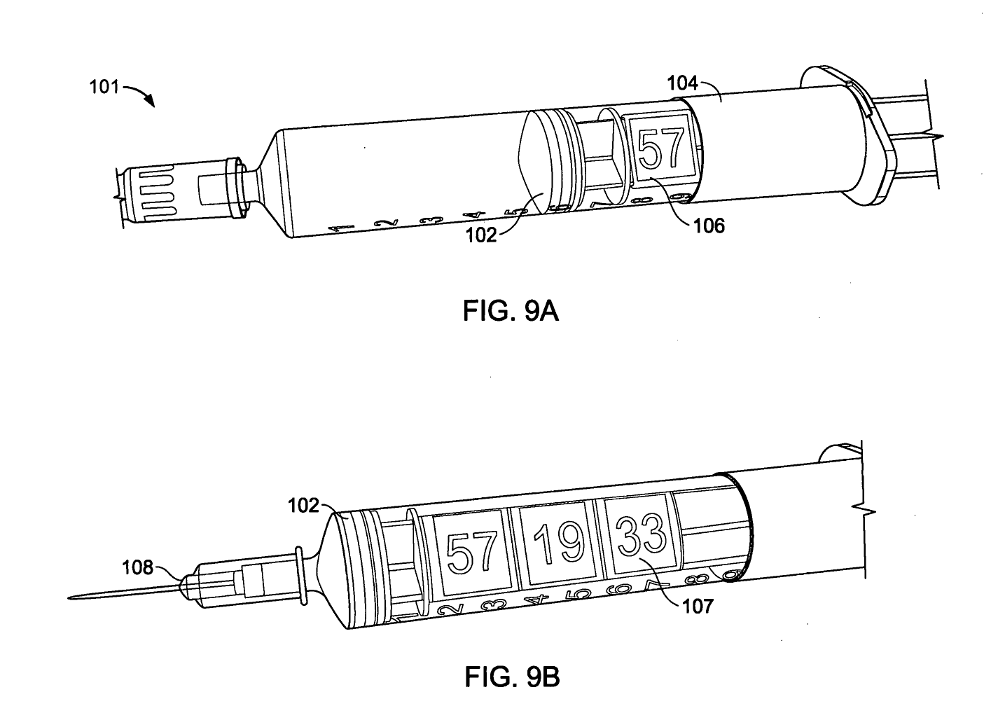

FIGS. 8A to 8C show a blister pack 84 in a circular configuration. FIGS. 8A and 8C show top views whereas FIG. 8B shows a side view. The main body 89 of the blister pack includes a plurality of blisters 85, which each typically contain one pill, but can contain multiple pills. The main body is marked with a series of numbers (87). A movable (e.g., rotatable) disk 90 is attached to the main body 84 by a central axle 88 such that the disk may rotate with reference to the main body, but may not separate from the main body in normal use. This rotation can be achieved in various ways, for example, by flanges on either end of the central axle such that the flange diameters both exceed the respective diameters of central holes in the main body 89 and the movable disk 90. The disk 90 is marked with a series of letters 87. As shown in FIG. 8A, the number “58” (reference 87) is aligned with the letter “X.” In FIG. 8C, the movable disk 90 is rotated such that the number “58” is aligned with the letter “R.”

FIGS. 8D to 8F show a blister pack 92 in a rectangular configuration with a dispenser code 93. FIGS. 8D and 8F show top views whereas FIG. 8E shows a side view. The main body 99 of the blister pack includes a plurality of blisters 95, which each contain one or more pills. The main body 99 is marked with a series of letters (94). A movable sleeve 100 partially envelops the main body 99, as best seen in FIG. 8E, such that the patient may slide the sleeve linearly with reference to the main body. The sleeve 100 is marked with a series of number 96. As shown in FIG. 8D, the number “12” is aligned with the letter “R.” In FIG. 8F, the sleeve 100 is moved such that the number “38” (reference numeral 96c) is aligned with the letter “R” (94a).

The embodiments shown in FIG. 8 can be varied in a limitless number of ways. Specifically contemplated are variations in which the main body (cf. 89 and 99) envelops or even obscures from view most of the movable component (cf. 90 and 100) or vice-versa; e.g., in some embodiments, the indicia or symbols on the movable component are visible only through a small opening or window in the main body, and in some embodiments only after the movable component has been moved with respect to the main body.

Syringe

As shown in FIGS. 9A and 9B, the medication dispenser can be in the form of a standard syringe 101 with a plunger 102 and optional needle 108. As shown in FIG. 9B, affixed to the plunger is a series of markings, e.g., numbers 107, which are visible once the plunger has been depressed. As shown in FIG. 9A, however, an opaque area 104 on the syringe conceals some of the numbers before the plunger is depressed; only one number “57” (106) is visible in this state. This number could be, for example, the dispenser identification code.

Thus, a user must depress the plunger 102, and most likely administer the drug, to see the markings from which to derive the reward code. In some instances, the markings, e.g., numbers, can be the reward code. In other instances, the reward code may be only one or two of several numbers, letters, or symbols, that are revealed once the plunger of the syringe is depressed.

A number of alternative embodiments are possible, for example, letters, numbers, and/or symbols could be printed on the syringe body and associated or aligned with particular numbers on the plunger once the plunger has been depressed, in a similar fashion as described for the blister packs (see above).

Methods of Determining Rewards

The new systems can provide rewards to users according to various methods of determining the timing and value of a given reward for a given compliance event. In general, the systems can provide users a reward report that recites an intermittent or a continuous reward. Thus, the reward may be intermittent with a fixed or varying value, or constant (e.g., a reward for each call) with a varying value, e.g., the value could be a chance to obtain a larger reward, e.g., a lottery ticket, or a small value such as one dollar, or one or more “points” or coupons, which can be combined with other points or coupons to obtain a larger reward. Various methods and algorithms, including Dynamic Intermittent Reward (DIR) and Tabular Intermittent Reward (TIR) methods described in further detail below, are used to determine the exact timing and value of the intermittent rewards.

In some embodiments, the medication dispensers described herein can also be used with a continuous reward schedule, whereby each contact with the contact center results in a reward having the same value. For example, a patient may use a specially marked dispenser and contact a control center, e.g., once a day, when they take their medication, and receive a reward, e.g., of $1, $2, or $5, or a coupon or point value that can be combined with other coupons or points, for each valid contact.

Intermittent Rewards

The new medication dispensers can be used in a system that provides an intermittent reward report, i.e., the patient gets a reward at some random interval, or the patient gets a reward each time, but the value is a randomly assigned value. These methods can be implemented using any known random number or pseudo-random number generator, or a predetermined table or mathematical function. Alternatively, the reward can be a chance to obtain a larger reward, e.g., a lottery ticket or other ticket for a game of chance, so that a patient who contacts the center consistently at the appropriate times obtains more lottery tickets, and thus has a greater chance of obtaining a reward.

In other embodiments any of the dispensers can be used with a fixed-ratio intermittent reward schedule, whereby a fixed number of contacts with the contact center results in a reward. For example, a patient may use a specially marked dispenser and contact a contact center when it is time to take their medication, e.g., once a day, but not more often, and receive a fixed reward of $50 for every tenth contact.

Dynamic Intermittent Rewards (DIR)

In some embodiments, the new automated contact center includes a Dynamic Intermittent Reward (DIR) system built on behavioral science and medical informatics. The principal aim of DIR is to achieve optimal behavior, e.g., medication compliance and keep the system cost-effective. The general principles of operation are as follows.

DIR is defined as any method of intermittent reward wherein the value or frequency of reward, or both, are inversely correlated with an individual's frequency of the desired behavior(s). For this definition, “inversely correlated” includes any inverse correlation, whether linear, nonlinear, univariate, or multivariate. A technical definition of such an inverse correlation is one that yields a Spearman's rank correlation coefficient of less than 0, e.g., −0.3, −0.5, or −0.7. For example, in some embodiments this correlation coefficient approaches or equals a value of −1. The Spearman's rank correlation coefficient is well understood and can be easily calculated by standard methods by anyone skilled in the art. Thus, based on the description herein various functions that yield such a correlation coefficient can be determined, and several exemplary functions are described herein.

A corollary of the DIR definition is that each time an individual performs a desired behavior, DIR tends to apportion more reward, or is more likely to apportion a reward, to an individual who has previously performed a desired behavior less frequently. Conversely, each time an individual performs a desired behavior, DIR tends to apportion less reward, or is less likely to apportion a reward, to an individual who has previously performed a desired behavior more frequently. This new method, which at first glance may appear counter-intuitive, gives rise to a number of surprising and useful properties.

Some implications of DIR are as follows:

(1) Each time an individual with an inferior behavior pattern (i.e., less frequent desired behavior) performs a desired behavior, he or she tends to receive more incentive, or is more likely to receive an incentive, and the amount of incentive or likelihood thereof is positively correlated with the degree of behavior inferiority.

(2) Each time an individual with a superior behavior pattern (i.e., more frequent desired behavior) performs a desired behavior, he or she tends to receive less incentive or is less likely to receive an incentive, and the amount of incentive or likelihood thereof is inversely correlated with the degree of behavior superiority.

(3) When an individual modifies his or her behavior by performing a desired behavior either more or less than before, the reward value or frequency (or both) is updated in an iterative fashion, based on the rules outlined above.

(4) When an individual with an inferior behavior pattern receives a substantial or increased reward, he or she will tend to respond with an improved behavior pattern. DIR then reduces the reward value or frequency (or both) in an iterative fashion, based on the rules outlined above.

(5) Individuals with inferior behavior patterns tend to receive increased incentives each time they perform a desired behavior. At the same time individuals with superior behavior patterns incur lesser reward expenditures each time they perform a desired behavior.

(6) Given the phenomenon in (5), DIR is particularly useful in scenarios where individuals may have other motives to perform a desired behavior.

It is helpful to contrast DIR with methods that apply intermittent rewards as well as methods that apply continuous rewards. Methods of intermittent reward (such as a lottery) do not allow different, tailored reward frequencies to help patients who need it most. Continuous reinforcement, on the other hand, involves fixed rewards, for example, $3 per dose. To reward patients sufficiently with either of these approaches is likely to be prohibitively expensive. DIR, on the other hand, motivates each patient individually at reasonable costs for the overall system.

The essential defining elements of DIR include: (1) a means to detect a desired behavior in an individual; (2) a record of the individual's desired behavior(s) over a period of time; (3) one or more mathematical functions to dictate when a reward should be administered or of what value the reward should be (or both), wherein (4) the reward frequency or amplitude is inversely correlated to the recorded frequency of the desired behavior.

The DIR system uses mathematical functions to modulate reward frequency and value. For a patient with lower compliance, DIR tends to provides additional “help” to the patient until compliance improves. Under these circumstances, this elevated cost of rewards is counterbalanced by the patient's low compliance rate, which means he or she “misses out” on many rewards. As the patient responds to the rewards and compliance improves, the payoff will return to normal levels. At the same time, a patient with more compliance tends to have more opportunities to win, so the system may, if one desires, be designed to treat patients with different behavior patterns differently yet equitably. Any time a patient's compliance diminishes, DIR comes to his or her aid with an extra rewards. This time around, however, the patient's pre-conditioned behavior is expected to return rapidly to peak levels. To render it more interesting to the patient, DIR may include other features, such as random “noise” and “super-rewards” such as an automobile or vacation.

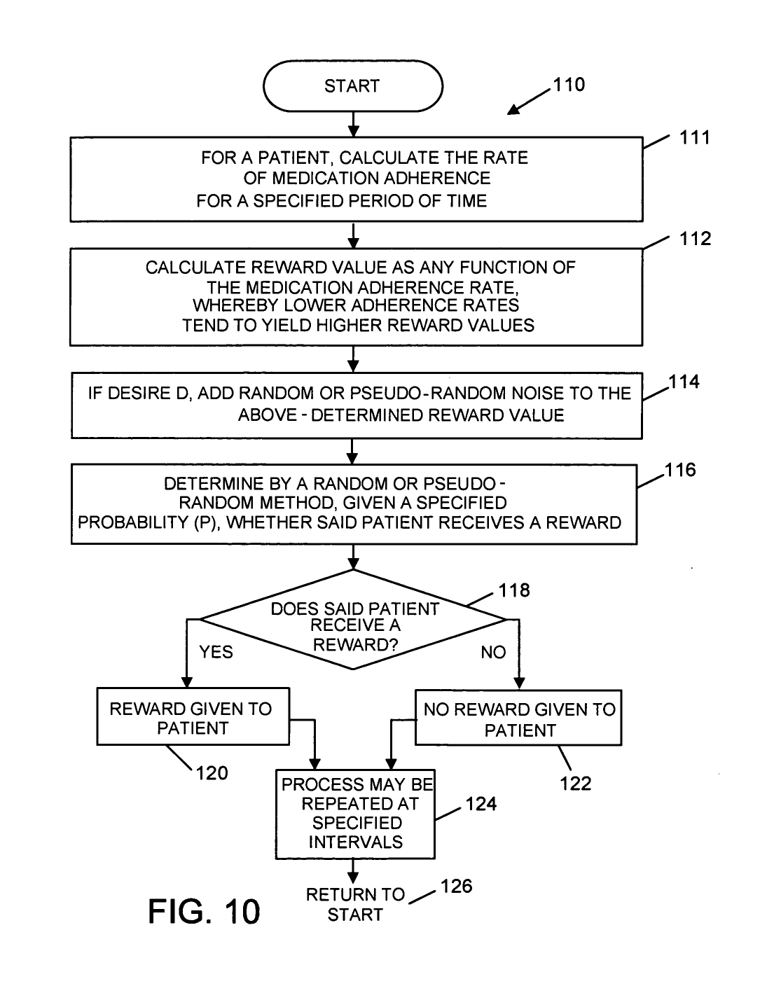

An exemplary method to implement DIR is described below and summarized in FIG. 10. The method 110 is conducted each time a patient is eligible for a reward (or it may be conducted ahead of time, with the results stored for later use). Steps 111, 112, and 114 relate to the assignment of a Reward Amplitude, and step 116 relates to the assignment of Reward Given, i.e., whether the patient will receive a reward on that particular occasion. FIG. 10 shows that step 116 occurs after step 111, 112, and 114, but it should be understood that step 116 could also be conducted before or in parallel with the aforementioned steps.

As shown in FIG. 10, a patient rate of medication adherence (or compliance rate) is calculated (111). A representative method would be to divide the number of adherence events during a given time period (e.g., the number of telephone calls to the Automated Contact Center) by the number of expected compliance events, e.g., doses prescribed according to a prescription for the given medication dispenser during the same time period. For example, an adherence rate can be calculated as the number of doses taken on-time over the last seven days divided by the number of doses prescribed for the last seven days. Other methods for calculating a compliance rate are explained below.

The next step 112 is to calculate a reward value (“Reward Amplitude”) as a function of the compliance rate calculated in step 111. This method should tend, on a stochastic basis, to yield an inverse correlation between the adherence rate and the reward value. For example, the rate may be calculated as $100 multiplied by (1.1—adherence rate).

The next step 114 is to add “noise,” if desired, to the reward value. The purpose of this noise is to add an additional level of interest for the patient. For example, one may multiply the reward by the value R, whereby R is a uniformly distributed random number between 0 and 2.

The next step 116 is to determine whether the patient will receive a reward or not for the particular Adherence Event in question (“Reward Given” or “RG”). Detailed methods are explained below. For example, assume that the desired probability of a reward is 0.33 and further assume a uniformly distributed value RG between 0 and 1, whereby the patient will receive a reward if and only if RG<P. The output of step 116 is evaluated at branchpoint 118. If the patient is to receive a reward, the contact center announces and/or awards the reward (120), and the patient may repeat the entire method at specified intervals (124), returning (126) to the starting state. If the patient is not to receive a reward, it is announced and/or recorded (122), and the patient may repeat the entire process at specified intervals (124), returning (126) to the starting state.

One approach to implement DIR includes storing patient adherence information on a database, e.g., a computer-controlled electronic database. A patient's identity and daily record of medication use are stored in a standard database, e.g., on a computer. Any database (or even a simple variable array or flat text file) can be used, e.g., Microsoft SQL Server or Microsoft Access. A patient's record of medication use is preferably stored as a time-and-date record of a patient's contacts to an automated contact center, e.g., calls to a specified automated call center, or a patient's record of activity on a specified web site. Adherence information can also be acquired automatically by a wireless or non-wireless device made to monitor medication use and communicate that information to a database.

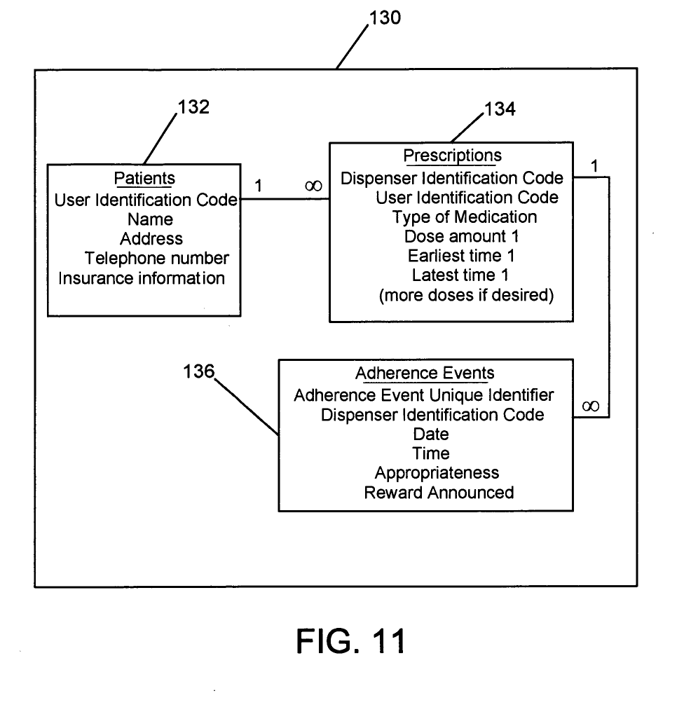

FIG. 11 shows an exemplary architecture of a database 130. The database contains:

1. A table called “Patients” 132, where each patient has his or her own record, and whereby each record contains a user identification code and other desired identifying information.

2. A table called “Prescriptions” 134, whereby each record contains a dispenser identification code, a user identification code, the type of medication prescribed, and an expression of the dosing schedule for the medication. In this example three variables are specified for each dose to be used in a 24-hour period: the dose amount, the earliest time of day, and the latest time of day. For example: Lipitor® 20 mg, earliest time 3 pm, latest time 11 pm. A twice-daily drug could be: Lopressor® (1) 25 mg, earliest time 6 am, latest time 12 pm; Lopressor® (2) 25 mg, earliest time 6 pm, latest time 11 pm. A single record in “Patients” may be related to one or many records in “Prescriptions.”

3. A table called “Adherence Events” 136, whereby each record corresponds to a single instance that the patient has contacted the contact center. Each record contains an adherence event unique identifier code, a dispenser identification code, a date and time, the reward announced (if any), and Appropriateness. The “Appropriateness” field, which may carry values such as “on-time” or “not on-time,” is determined by comparing the time in the “Adherence Events” record with the earliest and latest times of day in the related “Prescriptions” record, as would be obvious to anyone skilled in the art. A single record in “Prescriptions” may be related to one or many records in “Adherence Events.”

The patient adherence information can be analyzed to determine rewards as follows. For each Adherence Event, patient adherence information is analyzed by a computer program to determine if the patient obtains a reward and if so, of what value. Alternatively, these parameters can be calculated ahead of time and stored in anticipation of the patient's next Adherence Event(s). A suitable programming language is Visual Basic, but any suitable language can be used.

An exemplary system determines whether the patient receives a reward for a particular Adherence Event. First, a Reward Frequency (RF) is specified, where RF is between 0 and 1, inclusive. For example, RF=0.35 would correspond to a frequency of 35%. For a particular Adherence Event, a simple way to determine whether the patient receives a reward is by the following test:

IF (RAND( )<RF) THEN (Reward Given=yes)
ELSE (Reward Given=no)

where RAND( ) is a uniformly distributed pseudo-random number between 0 and 1. The patient receives a reward for the present Adherence Event if and only if Reward Given=yes.

With the above test, the actual frequency with which patients obtain rewards will only approximate RF. If a way is desired to make the actual frequency precisely the same as RF, many methods exist that are well-known to those skilled in the art.

The above tests can be modified so that the reward frequency is modulated depending on patient characteristics. For example, the following test would render a higher probability of providing a reward to a patient with lower adherence:

IF (RAND( )<RF/AR) THEN {Reward Given=yes};
ELSE {Reward Given=no};

where AR (adherence rate) is a number between 0 and 1 inclusive, and where 1 represents perfect adherence and 0 represents a complete lack of adherence (see below).

Any of several possible functions or series can be used instead of RAND( ) as a pseudo-random number generator. Such functions or series, while not random, can be designed to yield virtually unpredictable results for a patient. For example:

f(x)=TRUNC (π log x)

where TRUNC is the decimal portion of the expression in parentheses, log is the natural logarithm, and x is any real positive number. Furthermore, if desired, a function can be used to transform RAND( ) to a normally distributed parameter.

Alternatively, instead of a reward frequency, the intermittent nature of the reward can be expressed in terms of a “Reward Ratio” (e.g., one reward for every three Adherence Attempts) or “Reward Delay” (e.g., after winning, patient must register three more Adherence Events before winning again). Again, any of several possible functions or series could be used instead of RAND( ) as a pseudo-random number generator. For example:

Reward Delay (in days)=MOD(êx, maximum_delay)

Where MOD is the modulus of êx with maximum_delay as the divisor, êx represents “e” raised to the exponential power x, e is the base of the natural logarithm, x is any real number, and “maximum_delay” represents the maximum possible value returned by the above function.

If a patient is to receive a reward for a particular Adherence Event, it is also necessary to determine the amount of the reward. A hallmark of DIR is that the reward value depends in part (or in whole) on the patient's medication adherence.

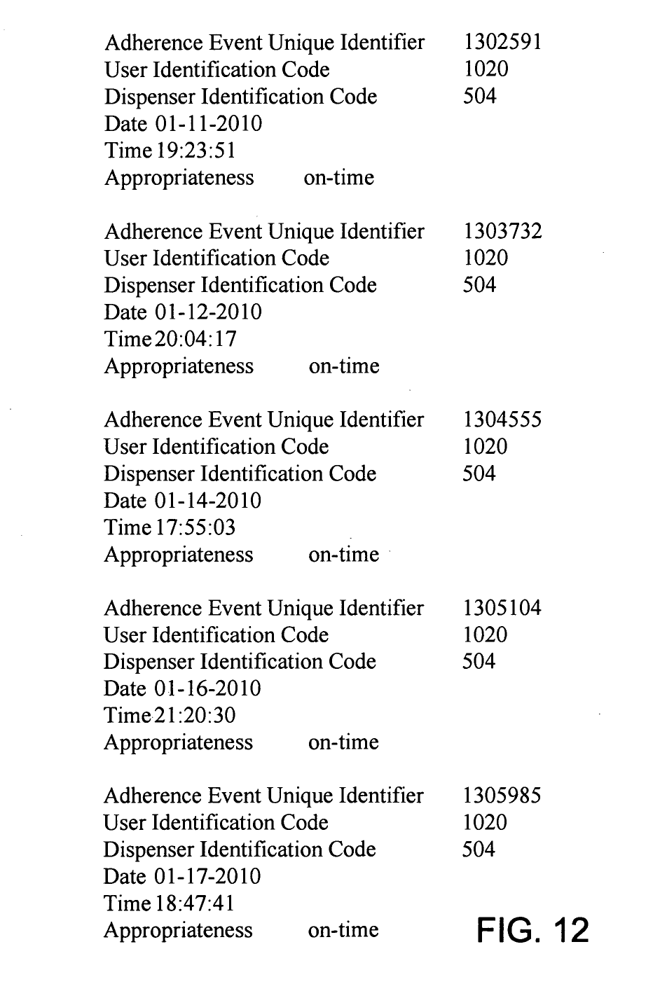

To make this determination, one or more adherence statistics must be calculated from the patient's Adherence Events. FIG. 12 shows a table of five exemplary records for all Adherence Events over the last 7 days for patient John Smith (user identification code 1020) for a prescription of Lipitor® 20 milligrams each evening (dispenser identification code 504).

To calculate adherence or compliance statistics it is first necessary to specify over what period of time adherence is to be calculated. For this example, referring to FIG. 12, adherence can be calculated for the 7-day period of Jan. 11, 2010 through Jan. 17, 2010. It is known from the “Prescriptions” table of the database that this prescription should be taken once every evening. From this information it follows that the Adherence Rate (AR) for the specified period is 5/7=0.7143 (i.e., the patient missed taking his medication on two days during the seven day period).

In this manner an Adherence Rate can be calculated for the past week, past month, or any other desired period of time. Note that the Adherence Rate, as it applies to the “past week,” will vary with each new day, as the definition of the “past week” is updated in a so-called “moving-window average.” The Adherence Rate will be a number between 0 and 1 inclusive. Likewise, Adherence Rates can be calculated for the week before the past week, the week before that, and so on.

In some embodiments more sophisticated functions can be used to incorporate records acquired over longer periods of time and if desired, weigh recent behavior more heavily than remote behavior. For example, suppose that a patient's record of medication use is kept for over 5 weeks, and an Adherence Rate (AR) is calculated for each of the last 5 weeks. Suppose the results are as follows:
Past 7 days 0.714
1 week prior 0.571
2 weeks prior 0.857
3 weeks prior 0.429
4 weeks prior 0.714

From the above values a preferred expression of adherence can be calculated by a geometric series as follows:

Adherence Index (AI)=AR(past 7 days)/2+AR(1 week prior)/4+AR(2 weeks prior)/8+AR(3 weeks prior)/16+AR(4 weeks prior)/32=0.714/2+0.571/4+0.857/8+0.429/16+0.714/32=0.656.

If sufficient data are available, the series may be expanded to include six or more terms. The Adherence Index (AI) will be a number between 0 and 1 inclusive. Just as an AI can be calculated based on present-day data, one can be calculated based on the data 7 days earlier, or for any other period in the past where data are available. In the above example, an AI calculated on the data 7 days earlier would be approximately: AI=0.571/2+0.857/4+0.429/8+0.714/16 . . . =approximately 0.598.

A skilled artisan will appreciate that other functions can be substituted for those above, with the same essential result of expressing a patient's adherence behavior over time.

Once a suitable expression of adherence is calculated, then the value of reward must be calculated. A useful approach is first to specify a Base Reward (BR), for example, $100. The final Reward Amplitude (RA) can then be calculated, for example as:

Reward Amplitude (RA)=BR*(1−AI)*RAND( )

where RAND( ) is a uniformly distributed (pseudo-)random number between 0 and 1. For example, if BR=$100, AI=0.656, and RAND( )=0.601:

Reward Amplitude (RA)=$100*(1−0.656)*0.601=$20.67.

Many other functions can be used to serve the purpose of determining reward amplitude from the above parameters. The essential requirements are (1) a base reward value; (2) AI or other suitable adherence measure; and (3) a random, pseudorandom, or other expression that varies (and whose distribution is not necessarily uniform). One or more of the above parameters can be compared or normalized with respect to other patients in the database, or for other time periods in the same patient. For example, it may be desirable to normalize RA or BR across all patients in the database, to control the overall value of rewards to be issued.

The principles of DIR dictate that the RA will tend, on a probabilistic basis, to be lower for higher values of AI. Likewise RA will tend to be higher for lower values of AI. Other expressions could be substituted for the (1−AI) in the above equation and still preserve the concept of DIR, for example (1/AI). Another preferred approach is |TA−A|, meaning the absolute value of the difference between TA and AI, where TA refers to a specific Target Adherence between 0 and 1.

In other embodiments, any of several possible fimctions or series can be used instead of RAND( ) as a pseudo-random number generator. Such functions or series, while not random, could be designed to yield interesting or difficult-to-predict results for a patient. For example:

f(x)=TRUNC(π log x)

where TRUNC is the decimal portion of the expression in parentheses, log is the natural logarithm, and x is any real positive number, e.g., the Dow Jones Industrial Average at market close the previous business day. Furthermore, a function can be used to transform RAND( ) to a normally distributed parameter.

In certain implementations, the Reward Amplitude can be further transformed or evaluated to serve other useful purposes. For example, it may be desirable to provide a higher reward to patients whose AI has recently improved. A method to accomplish this would add another term to the Reward Amplitude equation, such that:

Reward Amplitude (RA)=BR*(1−AI)*RAND( )*UF̂(10*(AI(today)−AI(7 days ago)))

Where UF (the “Uprise Factor”) is a coefficient typically between 1.0 and 3.0. With an Uprise Factor of 3.0, an improvement in AI over the past week would tend, on a probabilistic basis, to be associated with higher values of RA. With an Uprise Factor of 1.0, the expression UF(10*(AI(today)−AI(7 days ago))) would be equal to 1 and would not affect the Reward Amplitude.

In summary, DIR provides a new class of reward methods. The essential feature of these methods is that the value or frequency of the reward is inversely correlated with the frequency of the desired behavior. That is, when an individual performs a desired behavior, the individual who has previously performed the desired behavior less frequently will tend to receive more reward. This method may appear counter-intuitive at first glance, but it yields a number of useful and surprising properties to optimize behavior frequencies and reward costs.

Tabular Intermittent Reward (TIR)

Circumstances exist wherein it is desirable to calculate Reward Amplitude (or Base Reward) and Reward Given ahead of time for a plurality of anticipated conditions and store these values in a table (a “Static Table”) for later use. This approach, herein called Tabular Intermittent Reward (TIR), may be used with almost any method of intermittent reward, of which Dynamic Intermittent Reward is only an example. Particular advantages to this approach are that once a Static Table is generated, it is possible to determine with mathematical certainty under which condition(s) a user or patient will or will not receive a reward, and what the value of the reward will be. That is, for any person who possesses the Static Table (or necessary portion thereof), rewards will in theory be predictable and therefore non-random. At the same time, however, a Static Table makes it possible to vary rewards over time in a manner that is both varied and interesting to a user, allowing the excitement that users sometimes derive from intermittent rewards. The main overall advantage of using a Static Table is that it should, by virtue of having predictable outcomes, avoid the many regulatory and social issues occasioned by conducting a lottery or game of chance.

In certain embodiments, the Static Table can be made available to the user. Then, if a user (e.g., a patient) so desires, he or she can determine ahead of time whether a particular behavior (e.g., taking a pill) will result in a reward, and if so, how much. He or she can therefore make an informed decision whether said behavior would be “worth the effort” of participating. The invention further contemplates an optional service by which a participant could find out definitively whether he or she would win a reward on a particular occasion for performing a particular behavior. Such a system, while seemingly counter-intuitive, would be particularly useful for its ability to give the participant free choice, avoid an element of randomness, and in theory avoid perception and/or regulation as a sweepstakes or game of chance.

One way to implement TIR is to construct a table, herein called a “Static Table” which contains N+1 records, wherein N is any whole number, e.g., 10 or more. Each record lists the same three variables. The first variable, Adherence Bin, is an integer between 0 and N inclusive, whereby the records contain consecutive integer values from 0 to N, and whereby the record with Adherence Bin=0 corresponds to AI=0, and the record with Adherence Bin=N corresponds to AI=1.0. For example, if N=10, then the record with Adherence Bin=3 could correspond to the interval of 0.30≦AI<0.4, and the record with Adherence Bin=5 could correspond to the interval of 0.50≦AI<0.6.

This example assumes that the different values of Adherence Bin represent equal intervals of AI; however, this provision is not necessary and in some circumstances it may be desirable to let different values of Adherence Bin correspond to unequal intervals of AI. The second variable is Base Reward, as described above. The third variable is Reward Given (yes or no, as described above). For each value of Adherence Bin, Base Reward, and Reward Given values can be calculated based on AI=(Adherence Bin/N).

The table can be recalculated periodically, if desired, for example daily, weekly, or monthly. Each patient may have his or her own unique table, or the same table can be used for a plurality of patients. For each occasion one wishes to determine Base Reward and Reward Given, one refers to the table and refers to the record whereby Adherence Bin=INT (AI*N), whereby the function INT rounds the expression in parentheses down to the nearest integer. The corresponding values of Base Reward and Reward Given are then obtained from the same record.

Other types of Static Tables can be constructed. For example, Adherence Bin can be designed to depend on other parameters, such as AR, the day of the week, the patient's weight, or even relatively arbitrary values such as the temperature recorded at a given location every day at noon. The column of Base Reward could be replaced with a Column for Reward Amplitude. Furthermore, the Static Table would work equally well if it used the product of Base Reward (or Reward Amplitude) and Reward Given (let “yes”=1 and “no”=0) rather than the two factors individually. Alternatively, two or more tables could be created and used for different conditions (for example, a different table for each day of the week). The essential concept in all of these tables is that the Reward Given (yes or no) and/or some measure of reward size are calculated ahead of time, so that it is not necessary to a evaluate a random or pseudo-random expression in connection with a particular Adherence Event.

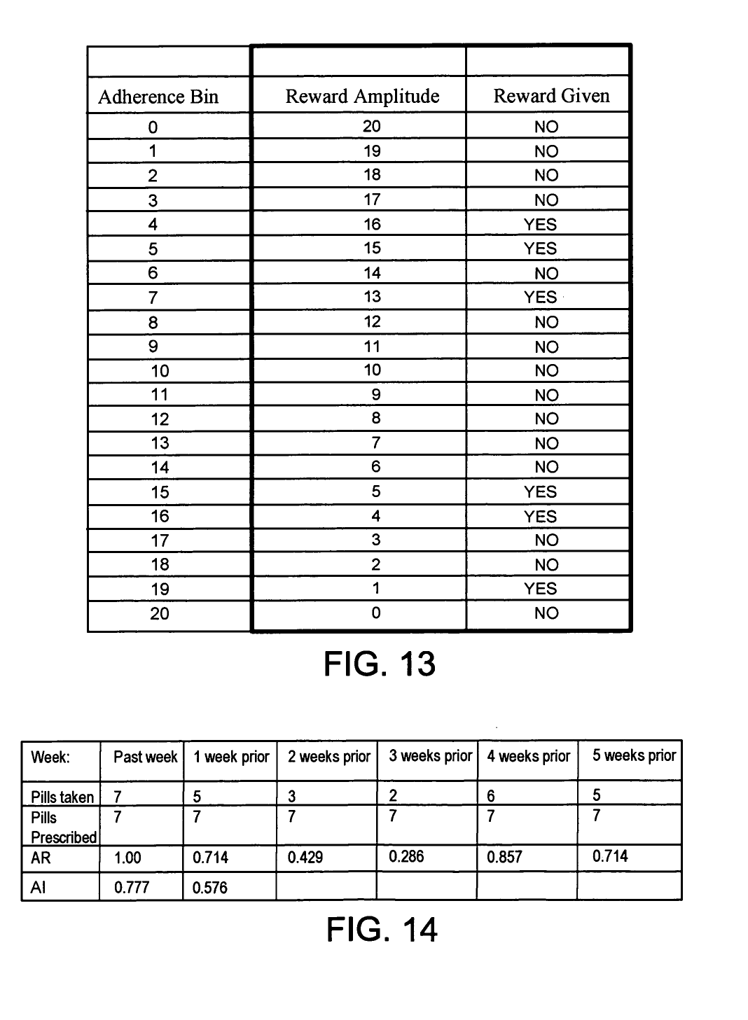

The following example illustrates how Tabular Intermittent Reward and Dynamic Intermittent Reward can be used together with respect to a patient's medication compliance. FIG. 13 shows an exemplary Static Table with 21 values for Adherence Bin and their associated values of Reward Amplitude and Reward Given. For this table, consistent with the concept of Dynamic Intermittent Reward, Reward Amplitude has been defined as:

Base Reward=20−Adherence Bin (where Adherence Bin is defined as above by the Adherence Index).

Based on this equation, the Base Reward is inversely correlated with the Adherence Index.

In this example Reward Frequency will be specified as 30%, and it happens that by the methods recited above, 6 of 21 Adherence Bins correspond to Reward Given=Yes. The other Adherence Bins correspond to Reward Given=No.

For this example let us now consider a patient Joe, who has taken his Lipitor® as shown in the table in FIG. 14, which provides the variables:
AI (past week)=0.777 and AI (previous week)=0.576.
Since AI=0.777 corresponds to Adherence Bin=INT (20*0.777)=15:
Reward Amplitude=5 and Reward Given=Yes.
For this example, let BR=$10, UF=1.5, and:

Reward Amplitude=BR*RM*BM*UF^{(10*(AI}^{₀}^{-AI}^{₁}⁾⁾

Therefore:

$$\begin{matrix}Reward Amplitude = \$10 * 0.421 * 1 * 1.5 \hat{} ( 10 * ( 0.777 - 0.576 ) ) \\ = \$4 .21 * 2.259 \\ = \$9 .51 .\end{matrix}$$

Variations on TIR

The TIR table, such as that shown in FIG. 13, can be replaced or updated repeatedly, for example every week. As described above for Dynamic Intermittent Reward, the contents of the table can be modified to promote particular behaviors. The tables, such as the one illustrated in FIG. 13, do not necessarily have to use 21 rows; they can use fewer rows (such as 11) or more rows (such as 101 or more). Nor does the table necessarily require an odd number of rows. Instead, the number of rows depends on how the possible values of AI (or another suitable parameter) are parceled into discrete intervals. All of the above variables can be calculated on a monthly basis or some other period of time, rather than weekly.

It should be noted that Tabular Intermittent Reward (TIR) and Dynamic Intermittent Reward (DIR) can be used separately or together. TIR can be used with many methods of intermittent reward.

In summary, TIR is a method of intermittent reward whereby the possible values of reward amplitude and frequency (or the product thereof) are pre-calculated and stored in a table, and whereby these values are later used to determine if a particular participant will receive a reward on a particular occasion, and if so, the value of the reward. The method makes it possible to reveal to participants, regulators, and/or the public a priori how rewards will be determined. It follows that once this information is revealed, all rewards provided by TIR methods are deterministic rather than random. At first glance the desire to reveal this information may appear counter-intuitive, but TIR methods are likely to have distinct ethical and regulatory advantages.

### Medication Compliance Improvement Systems

The new medication compliance improvement systems include the new medication dispensers and one or more control or contact centers, e.g., automated contact centers (ACC) that patients can contact via any form of communications network.

Automated Contact Center (ACC)

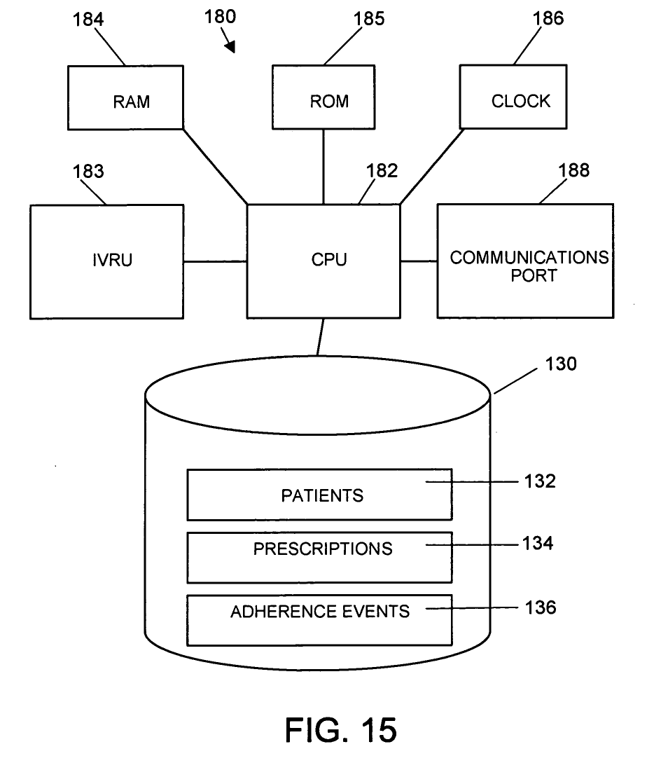

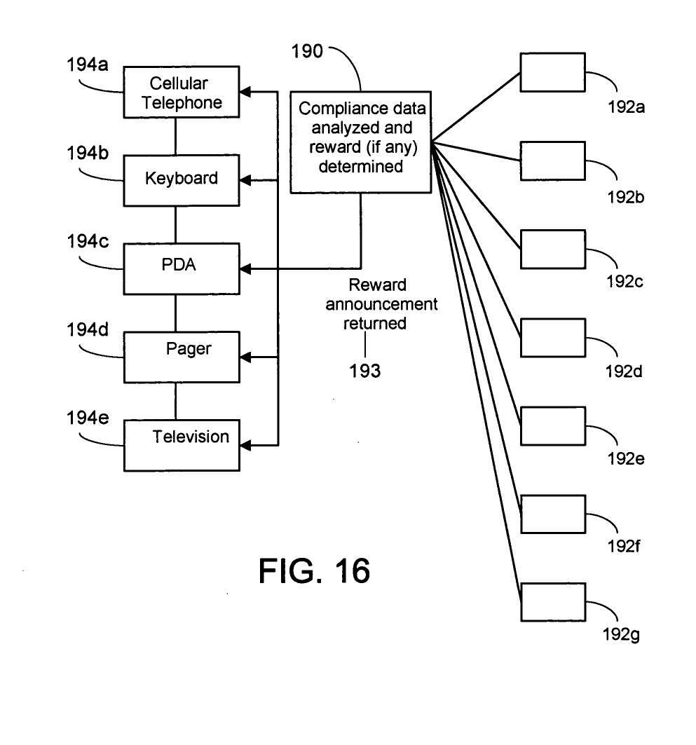

The new ACCs, as well as the various algorithms for DIR and TIR described above, can be implemented in hardware or software, or a combination of both. The invention can be implemented in computer programs using standard programming techniques following the method steps and figures disclosed herein. As shown in FIG. 15, the programs should be designed to execute on a programmable computer 180 each including at least one processor 182, at least one data storage system (including volatile and non-volatile memory and/or storage elements, e.g., RAM, 184 and ROM, 185), at least one communications port 188, that provides access for devices such as a computer keyboard (194b, FIG. 16), telephone (194a, FIG. 16), or a wireless, hand-held device, such as a PDA (194c, FIG. 16), and optionally at least one output device, such as a monitor, printer, or website. The central computer 180 also includes a clock 186 and an interactive voice response unit (“IVRU”) 183. These are all implemented using known techniques, software, and devices. The system also includes a database 130 that includes data, e.g., in the form of tables, for patient data 132, prescriptions data 134, and, in some embodiments, adherence event data 136.

Program code is applied to data input by a user (e.g., dispenser and user identification codes and reward codes-some of this information may be automatically determined by the system based on the user's telephone number using standard caller ID protocols) and data in the database, to perform the functions described herein and generate output information, such as whether a reward has been obtained and the value of the reward. The system can also generate inquiries and provide promotional messages to the user. The output information is applied to one or more output devices through the communications port 188 to devices such as a telephone, printer, or a monitor, or a web page on a computer monitor with access to a website.

Each program used in the new methods is preferably implemented in a high level procedural or object-oriented programming language to communicate with a computer system. However, the programs can be implemented in assembly or machine language, if desired. In any case, the language can be a compiled or interpreted language.

Each such computer program is preferably stored on a storage medium or device (e.g., RAM, ROM, optical, magnetic) readable by a general or special purpose programmable computer, for configuring and operating the computer when the storage media or device is read by the computer to perform the procedures described herein. The system can also be considered to be implemented as a computer-readable storage medium, configured with a computer program, whereby the storage medium so configured causes a computer to operate in a specific and predefined manner to perform the functions described herein.

The new ACCs and methods can be implemented using various means of data storage. For example, the individual user and prescription data files can be stored on a computer-readable medium (electronic apparatus readable medium) or in a computer or other electronic memory. The files can be transferred physically on recordable media or electronically, e.g., by email on a dedicated intranet, or on the Internet. The files can be encrypted using standard encryption software from such companies as RSA Security (Bedford, Mass.) and Baltimore®. The files can be stored in various formats, e.g., spreadsheets or databases.

As used herein, the term “electronic apparatus” is intended to include any suitable computing or processing apparatus or other device configured or adapted for storing data or information. Examples of electronic apparatus suitable for use with the present invention include stand-alone computing apparatus; communications networks, including local area networks (LAN), wide area networks (WAN), Internet, Intranet, and Extranet; electronic appliances such as a personal digital assistants (PDAs), cellular telephones, pagers and the like; and local and distributed processing systems.

As used herein, “stored” refers to a process for encoding information on an electronic apparatus readable medium. Those skilled in the art can readily adopt any of the presently known methods for recording information on known media to generate manufactures comprising the sequence information.

A variety of software programs and formats can be used to store dispenser, reward, user, and other data on an electronic apparatus readable medium. For example, the data can be represented in a word processing text file, formatted in commercially-available software such as WordPerfect® and MicroSoft® Word®, or represented in the form of an ASCII file, stored in a database application, such as Microsoft Access®, Microsoft SQL Server®, Sybase®, Oracle®, or the like, as well as in other forms. Any number of data processor structuring formats (e.g., text file or database) can be employed to obtain or create a medium having recorded thereon the relevant data and information.

By providing information in electronic apparatus readable form, one can routinely access the information for a variety of purposes. For example, one skilled in the art can use the data in electronic apparatus readable form to compare a specific set of data provided by a user with the information stored within a database. For example, search means are used to identify characters or series of characters in information provided by a user that match a particular reward, dispenser, or user code.

Communications Networks

Any communications network can be used including a telephone system, e.g., a mobile, wireless telephone system, an Internet-based system, or a closed wide area or local area network, e.g., within a hospital or clinic for use with patients. Although any communications network can be used, exiting telephone (either land lines or wireless) and the Internet provide useful choices to receive contacts from and transmit data to the users of the new automated contact center systems.

These communication networks can use either wired or wireless interfaces such as computers, telephones, PDAs, and other Internet access devices. In general to use a communications network such as the Internet or World-Wide-Web (WWW) an individual user runs a piece of software known as a Web browser, such as Internet Explorer® provided as part of the Windows operating system from Microsoft Corporation. The individual interacts with the browser to select a particular URL of the ACC, which in turn causes the browser to submit requests or data to the server identified in the URL. Typically the server responds to the request by retrieving or generating the requested page, and transmitting the data for the page back to the requesting individual. The content of the requested page may be either static or dynamic, whereby the latter can depend on mutable contents of a database or other information stored in memory or accessed from another device or network. In parallel, the server may receive, store, process, and/or retransmit data submitted by the individual to the server. The individual/server interaction is performed in accordance with the hypertext transport protocol (“http”) or other suitable protocol. This page is then displayed on the individual screen. The client may also cause the server to launch an application. The above protocols are useful not only for transmitting information between an individual and a server, but also between two servers, or between an automated internet-compatible device and a server, or between two automated internet-compatible devices.

### Promotional Applications of ACCs

The automated contact centers can provide many other benefits in addition to increasing patients' medication compliance. One such benefit is increased market share for drug manufacturers that participate in the new methods and systems. Since physicians are deeply concerned about non-compliance, they will choose brands of medication that use the new methods and systems to increase their compliance.

The automated contact centers can also be designed to provide a complementary system for drug manufacturers to communicate with consumers. For example, the systems can be designed so that each time a user calls the contact center they can listen to a brief promotional health message designed to encourage and/or educate the patient with respect to a healthy lifestyle in general, or about their particular disorder. Examples include “educe your risk of a heart attack. Please exercise every day.” Promotions can also be tailored to a specific brand name, companion product, company, pharmacy, or retailer. A special feature of the new medication adherence systems and methods is that the users voluntarily contact the automated system on a daily basis to find out if they will obtain a reward. Thus, because these systems never contact the users (but only receive voluntary contacts), they should raise no issues with respect to user, e.g., patient, privacy.

Messages can be tailored in a variety of ways. For example, if user's gender is stored in the database, the automated call center can be programmed to suggest a female vitamin formula to a woman and a male vitamin formula to a man. If the user's pharmacy of choice is stored in the database, the automated call center can be programmed to announce promotions specific to the patient's pharmacy. If the patient's zip code is stored in the database, the automated call center can be programmed to announce, for example, “Be sure to stay cool and drink plenty of water” to a patient in Phoenix, Ariz. in July, but to announce “When outdoors, dress warmly and watch out for frostbite” to a patient in Madison, Wis. in February.

In addition, the systems will provide direct reminders to patients to refill their prescription when the patients contact the automated center. This is a promotional opportunity not only for the drug manufacturers, but for drug stores and pharmacies as well. For example, the systems can be designed to determine when a patient is due for a refill. As an exemplary way to implement such as system, each “Prescription” record in the database (134, FIG. 11) could also include the fields “Number of doses in dispenser” and “Number of doses used.” Each time the patient contacts the ACC for a prescription, the value of “Number of doses used” is incremented by one. When the “Number of doses used” is greater than or equal to a specific number of doses (e.g., “Number of doses in dispenser” minus seven), the ACC will offer to forward a refill request to the patient's pharmacy automatically, with the patient merely pushing a button on a telephone keypad or a computer keyboard or saying “yes” to an inquiry by the system as to whether the patient would like to refill his or her prescription.

As a further useful feature, when the “Number of doses used” is greater than or equal to “Number of doses in dispenser,” the database can determine the associated dispenser to be “invalid” (e.g., 16, FIG. 3) and not allow the patient to determine a reward code (e.g., 18, FIG. 3). This will provide further incentive for the patient to refill his or her medication in a timely fashion.

These promotional methods always target an appropriate and voluntary audience. Patients who call the center are already taking the relevant medication and are actually interested in hearing about the drug and their condition. Moreover, since the contact is voluntary, patients are much more likely to be receptive to and persuaded by messages they receive from the contact center. See, e.g., Festinger, A Theory of Cognitive Dissonance, Stanford, Calif.: Stanford Univ. Press (1957). Furthermore, because these systems need not initiate contact with patients (but only require voluntary contact from patients), they should raise no issues with respect to patient privacy.

### EXAMPLES

The following examples are not to be construed as limiting the claimed invention.

### Example 1

### Clinical Trial Using an Intermittent Reward

A clinical trial was conducted to test whether a specially marked bottle, telephone contact center, and modest intermittent rewards would combine to improve patient compliance. The study design was a randomized crossover trial involving two phases: a Control phase and an Intervention phase. The drug regimen was a twice-daily placebo pill.

Study participants were recruited in person from community centers. The study included 12 volunteers (ages 40-65; 4 women, 8 men) of different socioeconomic status. The study protocol was explained to each participant, who then provided written informed consent. Each participant was then randomized to begin with either the Control phase or the Intervention phase. Six participants began with the Control phase and 6 began with the Intervention phase. Upon completion of the first phase, participants “crossed over,” i.e. proceeded, to the other phase. Thus, each person participated in both phases and served as their own control.

During the Control phase participants received a user identification code, a pill calendar, and a bottle of 28 placebo (sugar) pills in a white plastic bottle marked with a phone number and user identification code. These participants were instructed to take one pill in the morning and one in the evening, to call the specified telephone number and provide their user identification code to an automated call center, and to mark on the calendar each time they took a placebo pill. No rewards were provided. After 14 days the pill bottle and calendar were collected from the participant, and any remaining pills in the bottle were counted.

During the Intervention phase participants received a user identification code, a pill calendar, and a bottle of 28 placebo (sugar) pills in a white plastic bottle marked with a phone number and user identification code. The bottle also displayed a plurality of markings (a series of numbers) inside the bottle from which the users could derive reward codes. The participants were instructed to take one pill in the morning and one in the evening, to call the specified telephone number and provide their user identification code to the automated call center, and to mark on the calendar each time they took a placebo pill. Each time participants contacted the automated call center, they heard information which allowed them to inspect the bottle for a reward code and receive reward(s) of different values if the reward code was present within the series of numbers inside their bottle. Reward codes were not always present, and thus rewards were provided on an intermittent basis. In addition, rewards varied from $1 to $75, with a tendency toward rewards of $1 to $4. Participants who did not call were unable to receive rewards. After 14 days the pill bottle and calendar were collected from the participant, and any remaining pills in the bottle were counted.

Results analyzed included the record of calls to the automated call center, the number of pills remaining in a bottle, and the calendars completed by participants. As independent measures of pill compliance, these three measures showed excellent agreement. Over the four weeks of the study the retention rate was 92% (11 out of 12), and 643 calls to the automated call center (“compliance events”) were recorded.

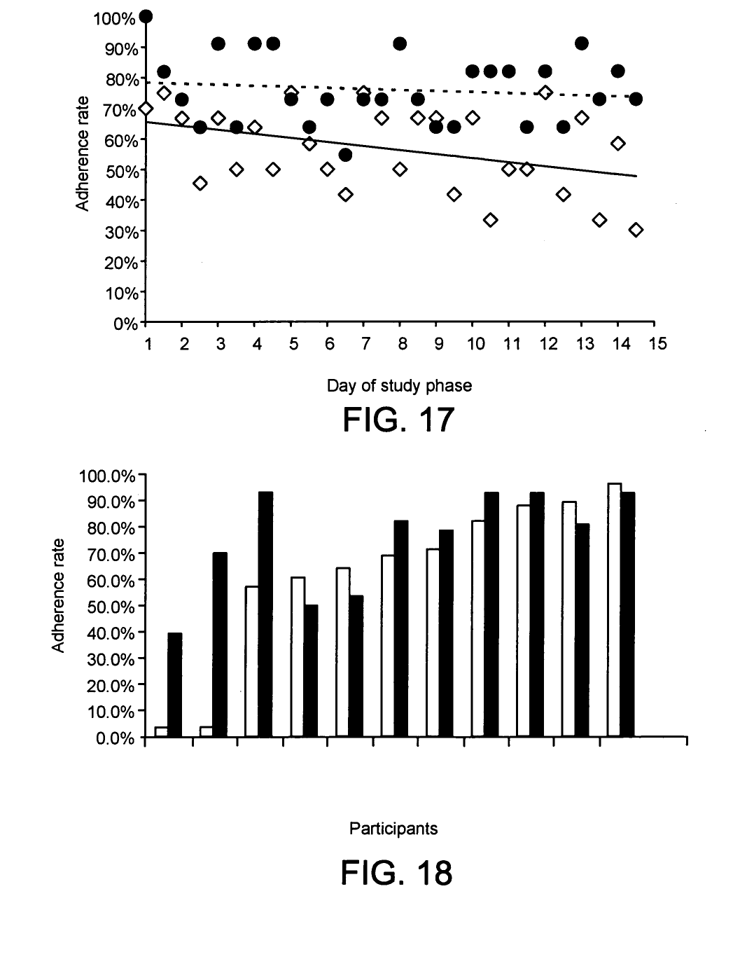

With intermittent reward, 233 doses were taken as scheduled and 78 were not (total of 311 doses). For the control, 187 doses were taken as scheduled and 145 were not (p<0.0001)(total of 332 doses). The results of this trial are shown in FIGS. 17 and 18.

FIG. 17 shows the mean adherence rates for each day of the Control (open diamonds, fit to solid line) and Intervention (solid circles, fit to dashed line phases of the study. Note that in the Control phase, adherence tended to diminish over 14 days. In the Control phase, however, adherence remained relatively sustained.

FIG. 18 shows mean adherence rates for each participant in the Control phase (open bars) and in the Intervention phase (filled bars). Participants with low adherence rates in the Control phase (at left) showed considerable increases during the Intervention phase. Participants with high adherence rates in the Control phase (at right), showed little or no difference during the Intervention phase. Although Dynamic Intermittent Reward (DIR) as described herein was not used in this trial, the results nevertheless support the utility of DIR, because behavioral changes occurred chiefly in response to rewards to participants with low baseline (Control phase) adherence rates; whereas there was little improvement (and little room for improvement) in participants with high baseline (Control phase) adherence rates.

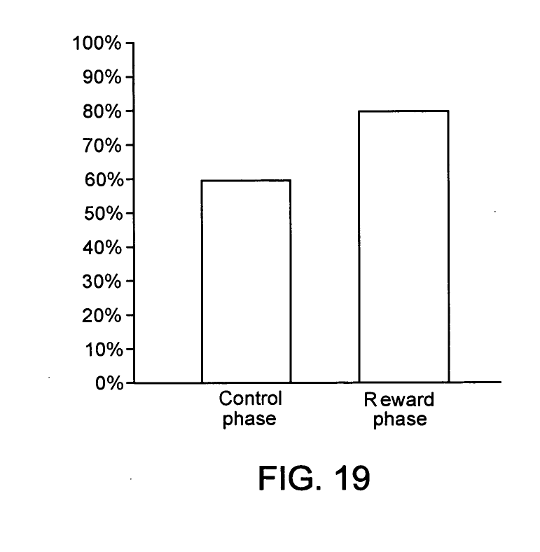

As shown in the bar graph of FIG. 19, the mean adherence rate for the group was about 60% for controls and about 80% with intermittent reward. This result corresponds to a 33% improvement in baseline compliance. Thus, the users were indeed motivated to call the contact center to discover if they had obtained a reward.

### Example 2

### Trial Using a Dynamic Intermittent Reward (DIR)

This example explains how DIR works and illustrates its unexpected and useful properties. The Department of Health offers intermittent rewards to the public to promote attendance at an a daily aerobics exercise class. A conventional method (not using DIR or any of the methods or systems described herein) would be to award $5 daily to 20% of individuals (randomly selected) who attend the aerobics class. Note that this method would not modify the reward in response to individual behavior.

Using DIR, the Department awards (to 20% of individuals, randomly selected) rewards of $20 minus $1 for each aerobics class an individual has attended over the previous 18 days. With this approach, a winning person with perfect attendance who has always loved aerobics would receive a reward of only $2. In contrast, a winning person with a record of poor attendance (or a person new to the aerobics class who has never attended a class) would receive a reward of $15 to $20. According to DIR, a winner of $15 to $20 is likely to be motivated to attend the next day, and the next day, and so forth. In time this person's attendance record would improve, and the rewards would diminish in a concomitant manner. In fact, this person's attendance is more likely to become or resemble “perfect attendance,” which would dictate rewards of as little as $2. Should this person's attendance then decline, the simple expression above would once more lead to an increased reward. The reward amounts would continue to respond to the patient's attendance record.

Eventually, the above example shows how under DIR, there will be a tendency for each person to reach an equilibrium with respect to reward value and behavior frequency. Reward values and frequencies can be modulated, even individually, to influence where this equilibrium occurs. A clear benefit of this approach is that the town's Department of Health is able to direct most of its reward funds to recruit and retain new people in the aerobics class, rather than compensate people who already attended the aerobics class for their own reasons. Another novel benefit of the system is that it provides more incentive to those people who need it most. Finally, another useful property is that DIR responds rapidly and automatically to a person's fluctuations in behavior in a way that will tend to return the person to a desired pattern of behavior.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A medication dispenser comprising
a container designed to contain one or more medication doses and comprising a first set of markings;
a movable component comprising a second set of markings and configured to move in relationship to the container to enable a user to align one or more markings of the first set of markings with one or more markings of the second set of markings and to enable the user to derive a reward code from one or more of the markings; and
a dispenser identification code associated with the dispenser.

2. The dispenser of claim 1, further comprising a user identification code associated with the dispenser.

3. The dispenser of claim 2, wherein the user identification code comprises an alphanumeric code stored on an electromagnetic device or on an electronic apparatus readable medium.

4. The dispenser of claim 2, wherein the user identification code comprises the user's telephone number.

5. The dispenser of claim 1, wherein one or more of the markings in the first or second sets of markings, or in both sets of markings, comprises a user identification code.

6. The dispenser of claim 1, wherein the container is a bottle comprising an opening and the movable component comprises a cap configured to seal the bottle, and wherein the first and second sets of markings are arranged so that they can be aligned by rotating the cap in relation to the bottle when the cap is placed over the opening of the bottle.

7. The dispenser of claim 6, wherein the first and second sets of markings are arranged so that when the cap is in a closed position, no marking on the bottle is aligned with a marking on the cap, and so that a marking in the first set can be aligned with a marking in the second set only when the cap is in an open position.

8. The dispenser of claim 1, wherein the container is a blister pack comprising a plurality of blisters each containing one or more medication doses.

9. The dispenser of claim 8, wherein the movable component is slideably attached to the blister pack such that the component can slide along a linear path in relation to the blister pack.

10. The dispenser of claim 8, wherein the movable component is rotatably attached to the blister pack such that it rotates around an axis of the blister pack.

11. The dispenser of claim 1, wherein the container is disc-shaped and has the first set of markings arranged around an axis, the movable component comprises a shell configured to rotate about the container and cover a portion of the container and has the second set of markings arranged around the axis, and wherein the first and second sets of markings are arranged so that they can be aligned by rotating the shell in relation to the container.

12. The dispenser of claim 11, wherein one or more of the markings on the container are covered by the shell when the dispenser is in a closed position.

13. The dispenser of claim 11, wherein the medication doses are inhalation doses of a medication in powder, liquid, or gaseous form.

14. The dispenser of claim 1, wherein the markings in the first set are numbers and the markings in the second set are letters, or vice versa.

15. The dispenser of claim 1, wherein the first and second sets of markings each comprises a random series of letters, numbers, or iconic symbols, or any combination of none, one, or more of letters, numbers, and iconic symbols.

16. A medication dispenser comprising
a container;
a movable component comprising one or more markings and configured to move in relation to the container to dispense a medication dose and to display the one or more markings after the dose has been dispensed to enable a user to derive a reward code from one or more of the markings; and
a dispenser identification code associated with the dispenser.

17. The dispenser of claim 16, wherein one or more of the markings on the movable component are covered by a portion of the container before the dose is administered.

18. The dispenser of claim 16, wherein the container comprises a syringe body and the moveable component comprises a plunger.

19. The dispenser of claim 16, wherein the container comprises a window through which one or more of the markings are visible after the dose is administered.

20. A method for improving medication adherence by a user, the method comprising
obtaining a dispenser identification code via a communications network to identify a medication dispenser comprising a marking or series of markings from which a reward code can be derived;
providing instructions to the user via the communications network to observe the identified medication dispenser and determine a reward code;
obtaining the reward code from the user via the communications network; and
providing the user with a reward report, wherein the reward report indicates whether a reward will be awarded, and if so, a reward value.

21. The method of claim 20, further comprising providing to the user a medicine dispenser associated with a dispenser identification code and comprising one or more sets of markings from which the reward code can be derived.

22. The method of claim 20, further comprising providing an automated contact center that a user can contact via the communications network and that provides automated instructions and the reward report to the user.

23. The method of claim 20, wherein the medication dispenser comprises a container comprising a first set of markings and a movable component comprising a second set of markings and configured to move in relationship to the container to permit a user to align one or more markings of the first set of markings with one or more markings of the second set of markings, and wherein the instructions comprise directions to the user to align a first mark in the first set of markings on the movable component with a second mark in the second set of markings on the container and to determine a reward code comprising a third mark aligned with a fourth mark.

24. The method of claim 20, wherein the user contacts the automated contact center with a telephone, the user's telephone number comprises a user identification code, and the automated contact center automatically determines the identity of the user based on the telephone number.

25. The method of claim 20, wherein the user must contact the center within a designated time period to be eligible to obtain a reward report.

26. The method of claim 20, further comprising obtaining the user's medication compliance history, and wherein the reward value, a frequency of a reward being awarded, or both are a function of the user's medication compliance history.

27. The method of claim 26, wherein the reward value, the frequency of a reward being awarded, or both are positively correlated with an improvement in the user's medication compliance history.

28. The method of claim 26, wherein the reward value, the frequency of the reward being awarded, or both are inversely correlated with the user's medication compliance history.

29. The method of claim 26, wherein the function includes a random or pseudorandom expression.

30. The method of claim 26, wherein the reward value, the frequency of the reward being awarded, or both are defined by a constant minus a measure of the user's compliance history over a specified period of time.

31. The method of claim 26, wherein the reward value, the frequency of the reward being awarded, or both are defined by a constant divided by a measure of the user's compliance history over a specified period of time.

32. The method of claim 26, wherein the reward value, the frequency of the reward being awarded, or both are defined such that, when correlated with a measure of the user's compliance history, the Spearman's rank correlation coefficient is less than 0.

33. The method of claim 20, wherein a reward value, a frequency of the reward being awarded, or both are selected from among a plurality of predetermined values, and wherein each predetermined value is associated with a particular measure of medication compliance.

34. The method of claim 33, wherein the reward value, the frequency of the reward being awarded, or both are defined by a constant minus, or divided by, a measure of the user's compliance history over a specified period of time.

35. The method of claim 33, wherein the reward value, the frequency of the reward being awarded, or both are defined such that, when correlated with a measure of the user's compliance history, the Spearman's rank correlation coefficient is less than 0.

36. A method for improving medication adherence by a user, the method comprising
identifying a medication user;
obtaining the user's medication compliance history for a specific medication;
determining whether a reward should be provided for a given compliance event as a function of the user's medication compliance history;
if a reward is to be provided, determining a reward value; and
providing the user with a reward report indicating whether a reward will be awarded and if so, the reward value.

37. The method of claim 36, further comprising calculating a medication compliance rate over a specific period of time based on the medication compliance history and determining whether a reward should be provided for a given compliance event as a function of the user's medication compliance rate.

38. The method of claim 36, further comprising providing to the user a medicine dispenser associated with a user identification code and obtaining the user identification code via a communications network to identify the user; and obtaining and storing a medication compliance history for the user and the specific medication.

39. The method of claim 36, wherein the reward value is determined as a function of the user's medication compliance history.

40. The method of claim 36, wherein the reward value, the frequency of a reward being awarded, or both are positively correlated with an improvement in the user's medication compliance history.

41. The method of claim 36, wherein the reward value, the frequency of the reward being awarded, or both are inversely correlated with the user's medication compliance history.

42. The method of claim 36, wherein the reward value, the frequency of the reward being awarded, or both are selected from among a plurality of predetermined values, and wherein each predetermined value is associated with a particular measure of medication compliance.

43. A system for improving medical adherence by a user, comprising
a medication dispenser of claim 1; and
a contact center comprising
a communications port,
a processor, and
an electronic apparatus readable medium configured to cause the processor to:
obtain the dispenser identification code via a communications network to identify the dispenser;
provide instructions to the user via the communications network to observe the medication dispenser and determine a reward code comprising one or more markings derived from the one or more sets of markings;
obtain the reward code from the user via the communications network; and
provide the user with a reward report, wherein the reward report indicates whether a reward will be awarded.

44. The system of claim 43, wherein the electronic apparatus readable medium is further configured to cause the processor to determine whether the user is communicating with the contact center at an appropriate time.

45. The system of claim 43, wherein the electronic apparatus readable medium is further configured to cause the processor to determine whether a reward is to be awarded, and if so, to determine a value of the reward.

46. The system of claim 45, wherein the electronic apparatus readable medium is further configured to cause the processor to determine the user's medication compliance history, and wherein the reward value, a frequency of a reward being awarded, or both are a function of the user's medication compliance history.

47. The system of claim 45, wherein the electronic apparatus readable medium is further configured to cause the processor to determine the user's medication compliance history, and wherein the reward value, a frequency of a reward being awarded, or both are selected from among a plurality of predetermined values, and wherein each predetermined value is associated with a particular measure of medication compliance.

48. The system of claim 43, wherein the communications port and processor are configured to enable a third party to determine the medical compliance of a patient.

49. The system of claim 43, wherein the electronic apparatus readable medium is further configured to cause the processor to report to the user that a medication refill is needed.

50. The system of claim 49, wherein the electronic apparatus readable medium is further configured to cause the processor to transmit a refill request to a medication dispensary.
